(19) United States
(12) Patent Application Publication
(10) Pub. No.: US 2026/0132159 A1
(43) Pub. Date: May 14, 2026

(54) GOLD CONTAINING COMPOUNDS AND METHODS OF USE

(71) Applicant: University of Kentucky Research Foundation, Lexington, KY (US)

(72) Inventor: Samuel G. Awuah, Lexington, KY (US)

(21) Appl. No.: 19/387,371

(22) Filed: Nov. 12, 2025

Related U.S. Application Data
(60) Provisional application No. 63/719,562, filed on Nov. 12, 2024.

(51) Int. Cl. C07F 9/6596; A61K 31/675

(52) U.S. Cl. CPC C07F 9/6596; A61K 31/675

(57) ABSTRACT

The presently-disclosed subject matter includes useful water-soluble gold containing compounds and compositions, and methods of making and methods of using such compounds and compositions.

### RELATED APPLICATIONS

This application claims priority from U.S. Provisional Application Ser. No. 63/719,562 filed Nov. 12, 2024, the entire disclosure of which is incorporated herein by this reference.

### TECHNICAL FIELD

The presently-disclosed subject matter relates to useful water-soluble gold containing compounds and compositions, and methods of making and methods of using such compounds and compositions.

### INTRODUCTION

Cancers characterized by elevated mitochondrial content and reliance on oxidative phosphorylation (OXPHOS) often evade standard-of-care (SOC) therapies, leading to therapeutic resistance, recurrence, and metastasis. Existing treatments largely overlook this metabolic vulnerability, leaving a gap in effective options for aggressive, treatment-refractory tumors. Compounds that target mitochondrial processes represent a biomedical need for addressing this gap, offering potential for the treatment of metabolic diseases, cancer, and inflammation, as well as serving as chemical probes to investigate mitochondrial function in vivo.

Current first-line cancer therapies, such as platinum-based drugs like cisplatin, are widely used for malignancies including triple-negative breast cancer (TNBC), but their clinical utility is often limited by resistance, toxicity, and insufficient potency. These drawbacks have driven exploration of alternative metal-based anticancer agents, particularly gold compounds, which provide a differentiated mechanism of action and are well tolerated in mammalian systems.

Beyond oncology, mitochondrial dysfunction has emerged as a key contributor to inflammatory disorders such as inflammatory bowel disease (IBD), where impaired mucosal repair and failure to maintain remission remain major challenges. Enhancing mitochondrial function could improve clinical outcomes by supporting intestinal health and promoting a balanced gut microbiome. Despite the therapeutic promise of gold-based compounds, progress has been constrained by synthetic difficulties and stability concerns, particularly in achieving water-soluble formulations. Accordingly, there remains a need for gold-containing compounds with improved bioavailability and robust chemical properties to unlock their full potential across diverse disease contexts.

### SUMMARY

The presently-disclosed subject matter meets some or all of the above-identified needs, as will become evident to those of ordinary skill in the art after a study of information provided in this document.

This Summary describes several embodiments of the presently-disclosed subject matter, and in many cases lists variations and permutations of these embodiments. This Summary is merely exemplary of the numerous and varied embodiments. Mention of one or more representative features of a given embodiment is likewise exemplary. Such an embodiment can typically exist with or without the feature(s) mentioned; likewise, those features can be applied to other embodiments of the presently-disclosed subject matter, whether listed in this Summary or not. To avoid excessive repetition, this Summary does not list or suggest all possible combinations of such features.

The presently-disclosed subject matter relates to useful water-soluble gold containing compounds and compositions, and methods of making and methods of using such compounds and compositions. Such compounds and compositions can be used, for example, for treating cancer and/or treatment of neurodegenerative disorders and/or modulating mitochondrial respiration and metabolism and/or modifying the intestinal microbiome to treat or protect against dysbiotic microbiomes, intestinal inflammation, colitis, inflammatory bowel disease (IBD) as well as other metabolic and inflammatory autoimmune disorders using compounds and compositions disclosed herein.

Compounds and compositions of the presently-disclosed subject matter target the mitochondrial gatekeeper Voltage-Dependent Anion Channel 1 (VDAC1) using gold-based compounds designed to selectively disrupt mitochondrial function in cancers dependent on oxidative phosphorylation (OXPHOS). A mechanistically distinct alternative to conventional chemotherapies and kinase inhibitors is thereby provided, addressing limitations observed in metabolically heterogeneous tumors such as triple-negative breast cancer (TNBC), melanoma, renal cell carcinoma, central nervous system malignancies, and radiation-resistant prostate cancer. Rare tumors, including Hürthle cell carcinoma, which exhibit excessive mitochondrial biogenesis, are expected to demonstrate unique sensitivity to this approach, thereby addressing an unmet need in oncology. Unlike standard-of-care agents that act through DNA damage or receptor signaling, compounds as disclosed herein are configured to exploit metabolic addiction and mitochondrial stress, offering a therapeutic avenue intended to expand the treatment landscape for both common and rare malignancies.

The presently-disclosed subject matter includes gold containing compounds that selectively target the mitochondrial protein VDAC1, an underexplored but essential metabolic hub in aggressive, therapy-resistant cancers. This approach is distinguished from standard-of-care agents such as platinum compounds, which act through DNA damage and are constrained by narrow therapeutic indices and systemic toxicity. In contrast, compounds as disclosed herein are designed to modulate mitochondrial bioenergetics and trigger immunometabolic cell death pathways, thereby avoiding genotoxic stress and providing a broader therapeutic window.

The presently-disclosed subject matter includes a compound having the following formula or a pharmaceutically acceptable salt thereof:

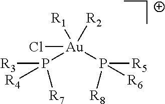

wherein R₁ and R₂, taken together with the gold (Au) to which they are bound, form a polycyclic moiety selected from the group consisting of:

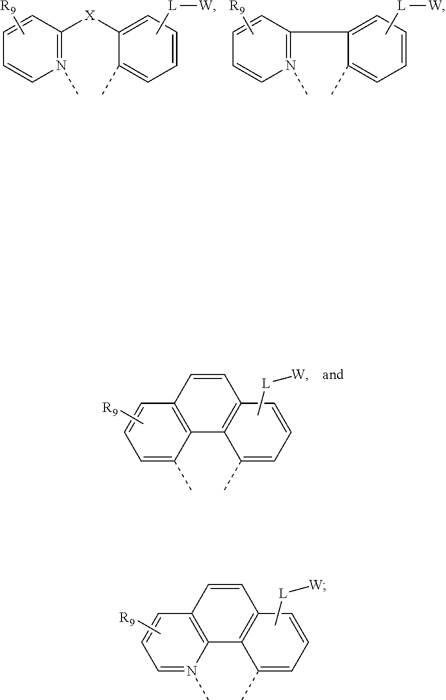

X is selected from the group consisting of CH₂, O, NH, C═O, C═NO-alkynyl, S, or aryl; R₃, R₄, R₅, R₆ are each independently selected from the group consisting of H, alkyl, and aryl, or R₃ and R₄ and/or R₅ and R₆, taken together with the phosphorus (P) to which they are bound, form a cyclic moiety; R₇ and R₈, together with the phosphorus (P) to which they are bound, form a structure selected from the group consisting of:

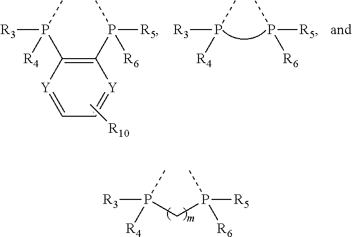

in which m is 1, 2, or 3; R₉ is H, alkyl, or aryl; R₁₀ is H, alkyl, aryl, or taken together with two of the atoms of the ring to which it is bound, forms a 6-membered ring; Y is C or N; L is a linker group; and W is a water soluble group.

In some embodiments of the compound, the polycyclic moiety formed by R₁ and R₂, taken together with the gold (Au) to which they are bound, is selected from the group consisting of:

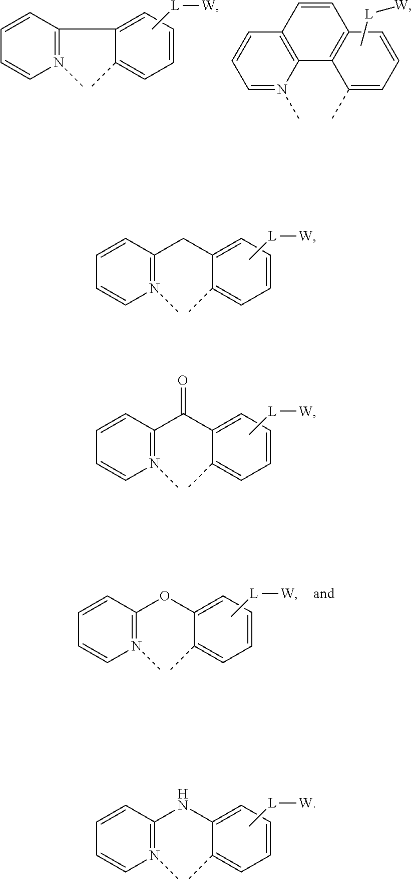

In some embodiments, the compound has a structure selected from the group consisting of:

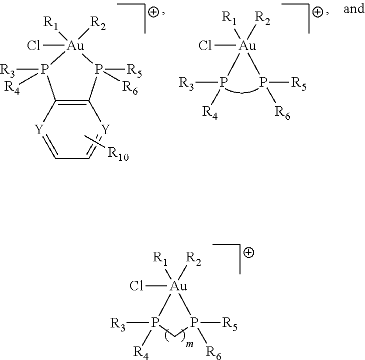

in which m is 1, 2, or 3; wherein R₁ and R₂, taken together with the gold (Au) to which they are bound, form a polycyclic moiety selected from the group consisting of:

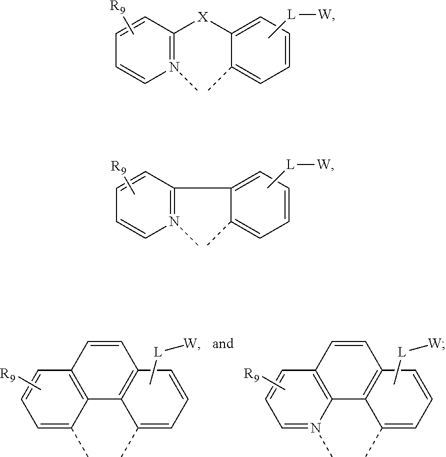

X is selected from the group consisting of CH₂, O, NH, C═O, C═NO-alkynyl, S, or aryl; R₃, R₄, R₅, R₆ are each independently selected from the group consisting of H, alkyl, and aryl, or R₃ and R₄ and/or R₅ and R₆, taken together with the phosphorus (P) to which they are bound, form a cyclic moiety; R₉ is H, alkyl, or aryl; R₁₀ is H, alkyl, aryl, or taken together with two of the atoms of the ring to which it is bound, forms a 6-membered ring; Y is C or N; L is a linker group; and W is a water soluble group; or a pharmaceutically acceptable salt thereof.

In some embodiments, the compound has the following structure:

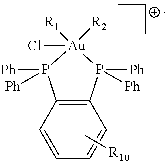

wherein R₁ and R₂, taken together with the gold (Au) to which they are bound, form a polycyclic moiety selected from the group consisting of:

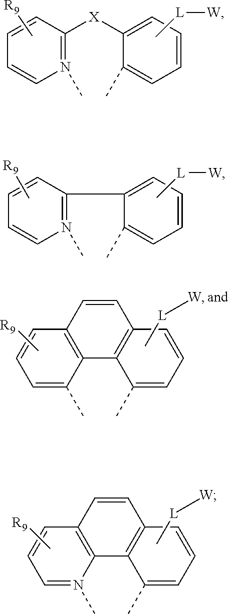

X is selected from the group consisting of CH₂, O, NH, C═O, C═NO-alkynyl, S, or aryl; R₉ is H, alkyl, or aryl; R₁₀ is H, alkyl, aryl, or taken together with two of the atoms of the ring to which it is bound, forms a 6-membered ring; L is a linker group; and W is a water soluble group; or a pharmaceutically acceptable salt thereof.

In some embodiments, the compound has a structure selected from the group consisting of:

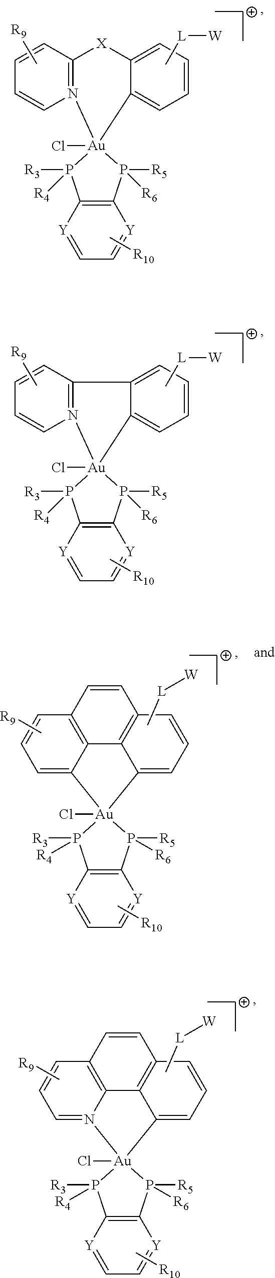

wherein X is selected from the group consisting of CH₂, O, NH, C═O, C═NO-alkynyl, S, or aryl; R₃, R₄, R₅, R₆ are each independently selected from the group consisting of H, alkyl, and aryl, or R₃ and R₄ and/or R₅ and R₆, taken together with the phosphorus (P) to which they are bound, form a cyclic moiety; R₉ is H, alkyl, or aryl; R₁₀ is H, alkyl, aryl, or taken together with two of the atoms of the ring to which it is bound, forms a 6-membered ring; Y is C or N; L is a linker group; and W is a water soluble group; or a pharmaceutically acceptable salt thereof.

In some embodiments, the compound has the following structure:

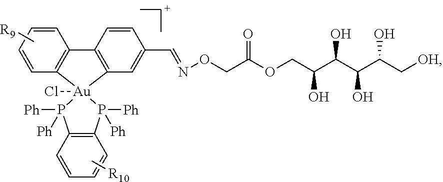

wherein R₉ is H, alkyl, or aryl; and R₁₀ is H, alkyl, aryl, or taken together with two of the atoms of the ring to which it is bound, forms a 6-membered ring.

In some embodiments, the compound has the following structure:

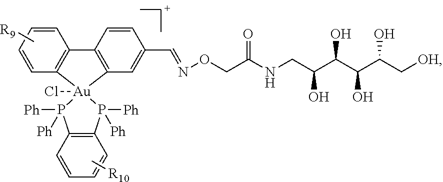

wherein R₉ is H, alkyl, or aryl; and R₁₀ is H, alkyl, aryl, or taken together with two of the atoms of the ring to which it is bound, forms a 6-membered ring.

In some embodiments, the compound has the following structure:

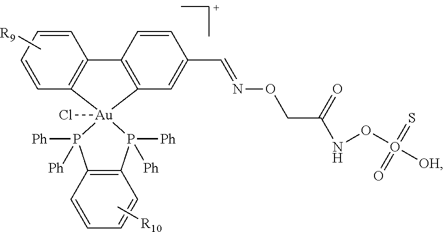

wherein R₉ is H, alkyl, or aryl; and R₁₀ is H, alkyl, aryl, or taken together with two of the atoms of the ring to which it is bound, forms a 6-membered ring.

In some embodiments, the compound has the following structure:

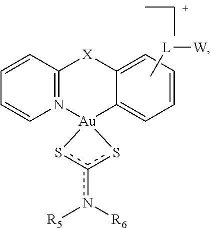

wherein X is selected from the group consisting of CH₂, O, NH, C═O, C═NO-alkynyl, S, or aryl; R₁ and R₂ are independently selected the group consisting of alkyl and substituted alkyl, or, R₁ and R₂, taken together with the N to which they are bound, form a piperidine or piperazine that is optionally substituted with Z; Z is selected from the group consisting of haloaryl and alkoxyaryl; L is a linker group; and W is a water soluble group.

In some embodiments of the compounds as disclosed herein, W is selected from the group consisting of

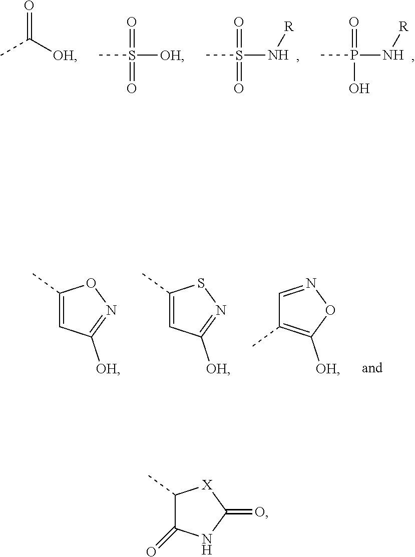

wherein R is selected from H, alkyl, and aryl.

In some embodiments of the compounds, W is selected from the group consisting of:

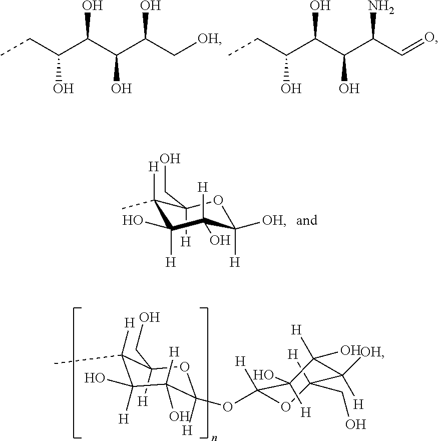

wherein n is 1-10.

In some embodiments of the compounds, W is selected from the group consisting of:

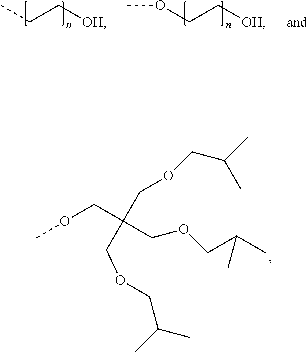

wherein n is 1-10.

In some embodiments of the compounds as disclosed herein, L is selected from the group consisting of:

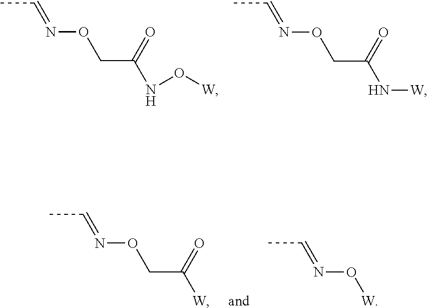

The presently-disclosed subject matter further includes methods of making a compound as disclosed herein.

The presently-disclosed subject matter further includes a pharmaceutical composition that includes a compound as disclosed herein and a pharmaceutically-acceptable carrier.

The presently-disclosed subject matter further includes use of a compound or composition as disclosed herein for treating a subject.

### BRIEF DESCRIPTION OF THE DRAWINGS

The features of the invention are set forth with particularity in the appended claims. A better understanding of the features and advantages of the present invention will be obtained by reference to the following detailed description that sets forth illustrative embodiments, in which the principles of the invention are used, and the accompanying drawings of which:

FIG. 1A-1C. Structure of AuPhos-89 (FIG. 1A), AuPhos89-sorbitol (FIG. 1B), and AuPhos89-glutamine (FIG. 1C).

FIG. 2A-2I. NCI DTP Screening results. Dose-response curves showing 5-dose summary of AuPhos-89 in the NCI-60 screen. Waterfall plot of GI50s of cell lines form the 5-dose testing of AuPhos-89.

FIG. 3A-3M. Target identification of AuPhos-89. FIG. 3A. Graphical representation of chemoproteomic workflow for AuPhos-89 target identification. FIG. 3B. In-Gel fluorescence of AuPhos-biotin pull down via click chemistry in MDA-MB-231 and NCM460 lysates. FIG. 3C-3J. AuPhos chemoproteomic analysis for target identification by LC-MS/MS and analysis showing volcanic plots, enrichment analysis, and GO terms of enriched proteins. FIG. 3K. Immunoblotting of pull down. FIG. 3L. CETSA validation indicates stabilization of VDAC1 by AuPhos-89. FIG. 3M. Quantification of CETSA blots.

FIG. 4. Expression analysis of VDAC1 and overall survival in human tumors establishes VDAC1 as viable therapeutic target in cancer.

FIG. 5A-5G. AuPhos-89 induces mitochondria dysfunction. FIG. 5A. Mitostress assay of PC3 cells treated with AuPhos-89 (200 nM) for 24 h along with mitochondrial complex inhibitors. FIG. 5B. Extrapolated oxygen consumption rate (OCR) for basal and spare respiratory capacity from (FIG. 5A). FIG. 5C. Extrapolated OCR for proton leak and ATP production from (FIG. 5A). FIG. 5D. Jess Western blot analysis of PC3 cells shows AuPhos-89 decreases expression of mitochondrial biogenesis (TFAM) and redox control enzymes. FIG. 5E. MitoSox red assay shows generation of mitochondrial ROS by AuPhos-89 at 200 nM for 24 h. FIG. 5F. Quantified mtROS production from (FIG. 5E). FIG. 5G. Jess Western blot analysis of PC3 cells shows AuPhos-89 decreases expression of mitochondrial fission 1 (FIS1) and dynamin-related protein 1 (DRP1).

FIG. 6. Stable isotope resolved metabolomics (SIRM) of AuPhos-89-treated PC3 3D-spheroids shows alteration of critical TCA cycle metabolites.

FIG. 7A-7G. Antitumor effect induced by AuPhos-89. (FIG. 7A) Impact of AuPhos-89 on the tumor volume of 4T1 (1 million cells inoculated, n=5). Unpaired t-test, *, D<0.05. (FIG. 7B) Weight of mice (n=3) following intravenous administration of AuPhos-89 and observed over 19 days. (FIG. 7C) Comparative in vivo efficacy study of AuPhos-89 and cisplatin. Impact of AuPhos-89 and cisplatin on the tumor volume of 4T1 (two million cells inoculated, n=5). Ordinary one-way ANOVA test, *, D<0.05. (FIG. 7D) Weight of mice following intraperitoneal administration of AuPhos-89 and cisplatin. (FIG. 7E) Excised tumor tissues and organs from (FIG. 7C). (FIG. 7F) Tissue biodistribution of AuPhos-89 in mice as determined by GF-AAS, which measures gold content. The compound was administered by intravenous injection and at indicated time points, tissues were collected after mice (n=3) were euthanized. (FIG. 7G) Hematoxylin and eosin (H&E) staining indicates reduced cellularity and proliferation in tumors treated with AuPhos-89. Liver metastasis is observed in control mice with no palpable metastatic lesions in treated mice.

FIG. 8A-8F. Efficacy studies of AuPhos-sorb. FIG. 8A-8C. Tumor volume and body weight of 4T1 inoculated Balb/c mice treated with 7 mg kg-1 AuPhos-sorb intraperitoneally FIG. 8D-8F. SUM159 cells inoculated Nu/J mice treated with 7 mg kg-1 AuPhos-sorb intraperitoneally.

FIG. 9A-9B. Pharmacokinetics (n=5) of AuPhos-89 in mice as determined by GF-AAS that measure gold content and confirmed by LC-MS/MS in Plasma (FIG. 9A) and tumor (FIG. 9B).

FIG. 10A-10D. Toxicity profile of AuPhos89-sorbitol treated mice assaying liver enzymes, blood and immune cells, body/organ weight at different doses (0.25, 2.5, 25 mg/kg PO).

### DESCRIPTION OF EXEMPLARY EMBODIMENTS

The details of one or more embodiments of the presently-disclosed subject matter are set forth in this document. Modifications to embodiments described in this document, and other embodiments, will be evident to those of ordinary skill in the art after a study of the information provided in this document. The information provided in this document, and particularly the specific details of the described exemplary embodiments, is provided primarily for clearness of understanding and no unnecessary limitations are to be understood therefrom. In case of conflict, the specification of this document, including definitions, will control.

The presently-disclosed subject matter includes compounds that are gold-based molecules, which have in vitro and in vivo utility with distinct modes of action. The compounds disclosed herein have utility as treatment agents with improved bioavailability relative to known gold-based compounds. Compounds as disclosed herein can be useful for conferring anti-cancer activity or for treatment of cancer, for treatment of neurodegenerative disorders, for modulating mitochondrial respiration and metabolism, for modifying the intestinal microbiome, for treatment or protection against dysbiotic microbiomes, for treatment of intestinal inflammation, for treatment of colitis, for treatment of inflammatory bowel disease (IBD), and for treatment of other metabolic and inflammatory autoimmune disorders.

The presently-disclosed subject matter includes a compound having the following formula or a pharmaceutically acceptable salt thereof:

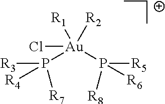

wherein R₁ and R₂, taken together with the gold (Au) to which they are bound, form a polycyclic moiety selected from the group consisting of:

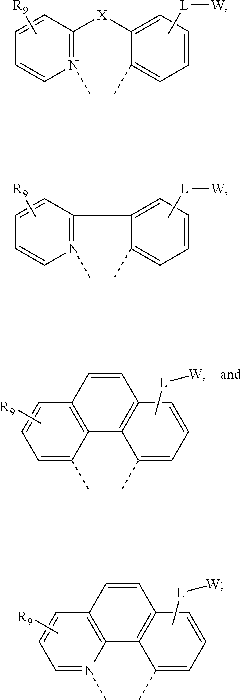

is selected from the group consisting of CH₂, O, NH, C═O, C═NO-alkynyl, S, or aryl; R₃, R₄, R₅, R₆ are each independently selected from the group consisting of H, alkyl, and aryl, or R₃ and R₄ and/or R₅ and R₆, taken together with the phosphorus (P) to which they are bound, form a cyclic moiety; R₇ and R₈, together with the phosphorus (P) to which they are bound, form a structure selected from the group consisting of:

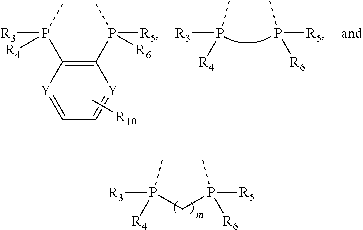

in which m is 1, 2, or 3; R₉ is H, alkyl, or aryl; R₁₀ is H, alkyl, aryl, or taken together with two of the atoms of the ring to which it is bound, forms a 6-membered ring; Y is C or N; L is a linker group; and W is a water soluble group.

In some embodiments of the compound, the polycyclic moiety formed by R₁ and R₂, taken together with the gold (Au) to which they are bound, is selected from the group consisting of:

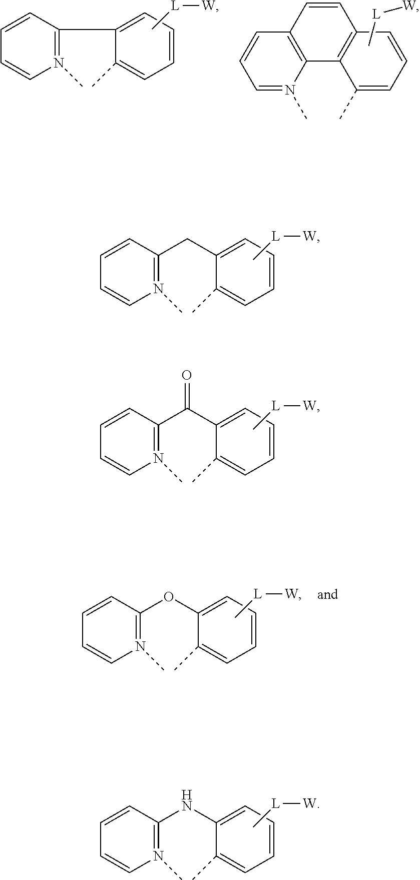

In some embodiments, the compound has a structure selected from the group consisting of:

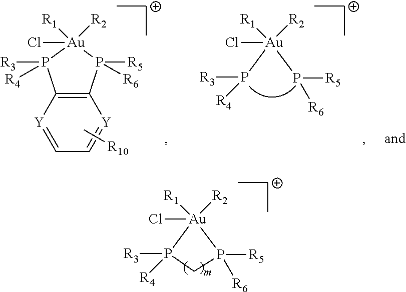

in which m is 1, 2, or 3; wherein R₁ and R₂, taken together with the gold (Au) to which they are bound, form a polycyclic moiety selected from the group consisting of:

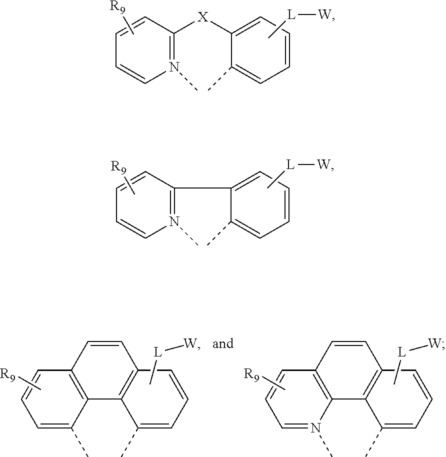

X is selected from the group consisting of CH₂, O, NH, C═O, C═NO-alkynyl, S, or aryl; R₃, R₄, R₅, R₆ are each independently selected from the group consisting of H, alkyl, and aryl, or R₃ and R₄ and/or R₅ and R₆, taken together with the phosphorus (P) to which they are bound, form a cyclic moiety; R₉ is H, alkyl, or aryl; R₁₀ is H, alkyl, aryl, or taken together with two of the atoms of the ring to which it is bound, forms a 6-membered ring; Y is C or N; L is a linker group; and W is a water soluble group; or a pharmaceutically acceptable salt thereof.

In some embodiments, the compound has the following structure:

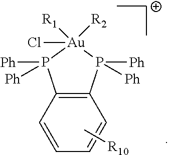

wherein R₁ and R₂, taken together with the gold (Au) to which they are bound, form a polycyclic moiety selected from the group consisting of:

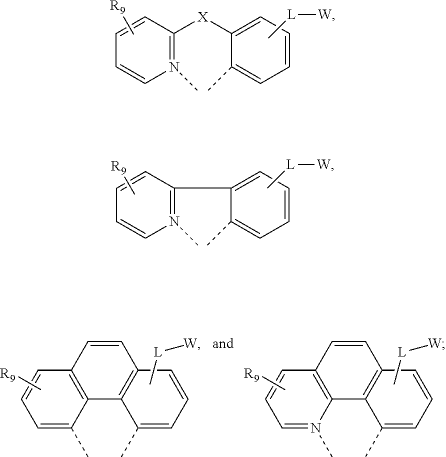

X is selected from the group consisting of CH₂, O, NH, C═O, C═NO-alkynyl, S, or aryl; R₉ is H, alkyl, or aryl; R₁₀ is H, alkyl, aryl, or taken together with two of the atoms of the ring to which it is bound, forms a 6-membered ring; L is a linker group; and W is a water soluble group; or a pharmaceutically acceptable salt thereof.

In some embodiments, the compound has a structure selected from the group consisting of:

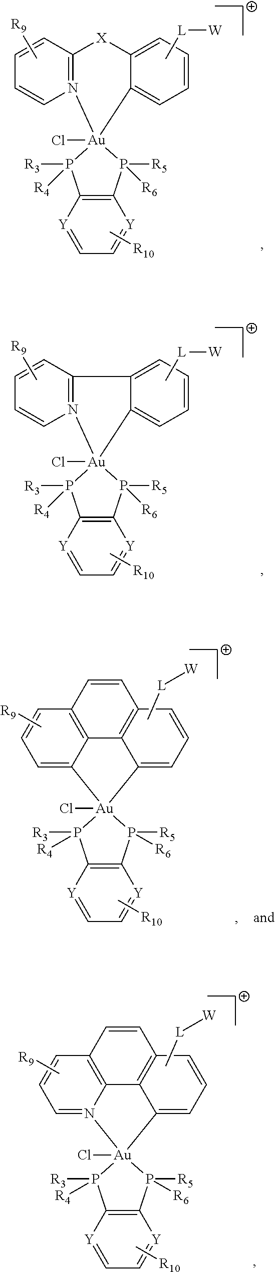

wherein X is selected from the group consisting of CH₂, O, NH, C═O, C═NO-alkynyl, S, or aryl; R₃, R₄, R₅, R₆ are each independently selected from the group consisting of H, alkyl, and aryl, or R₃ and R₄ and/or R₅ and R₆, taken together with the phosphorus (P) to which they are bound, form a cyclic moiety; R₉ is H, alkyl, or aryl; R₁₀ is H, alkyl, aryl, or taken together with two of the atoms of the ring to which it is bound, forms a 6-membered ring; Y is C or N; L is a linker group; and W is a water soluble group; or a pharmaceutically acceptable salt thereof.

In some embodiments, the compound has the following structure:

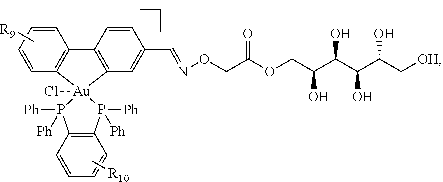

wherein R₉ is H, alkyl, or aryl; and R₁₀ is H, alkyl, aryl, or taken together with two of the atoms of the ring to which it is bound, forms a 6-membered ring.

In some embodiments, the compound has the following structure:

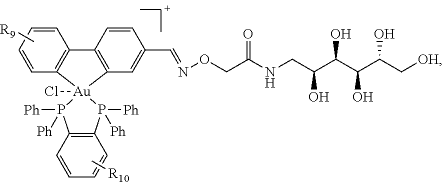

wherein R₉ is H, alkyl, or aryl; and R₁₀ is H, alkyl, aryl, or taken together with two of the atoms of the ring to which it is bound, forms a 6-membered ring.

In some embodiments, the compound has the following structure:

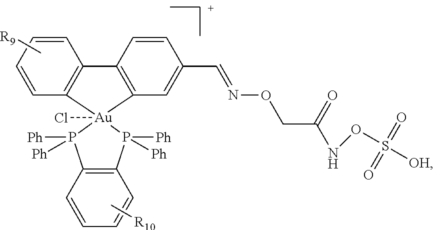

wherein R₉ is H, alkyl, or aryl; and R₁₀ is H, alkyl, aryl, or taken together with two of the atoms of the ring to which it is bound, forms a 6-membered ring.

In some embodiments, the compound has the following structure:

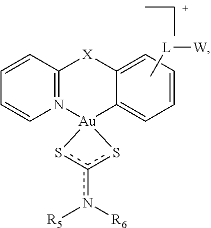

wherein X is selected from the group consisting of CH₂, O, NH, C═O, C═NO-alkynyl, S, or aryl; R₁ and R₂ are independently selected the group consisting of alkyl and substituted alkyl, or, R₁ and R₂, taken together with the N to which they are bound, form a piperidine or piperazine that is optionally substituted with Z; Z is selected from the group consisting of haloaryl and alkoxyaryl; L is a linker group; and W is a water soluble group.

In some embodiments of the compounds as disclosed herein, W is selected from the group consisting of:

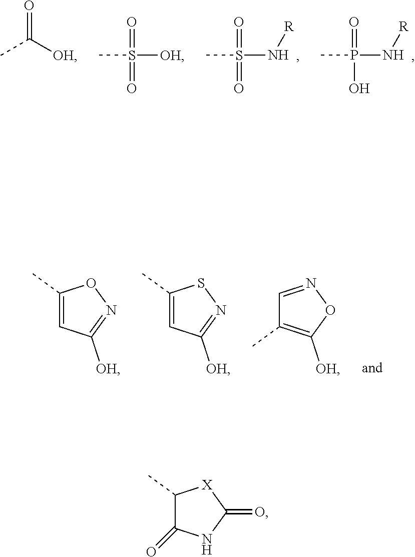

wherein R is selected from H, alkyl, and aryl.

In some embodiments of the compounds, W is selected from the group consisting of:

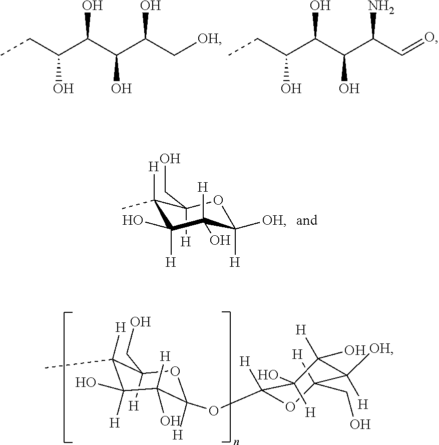

wherein n is 1-10.

In some embodiments of the compounds, W is selected from the group consisting of:

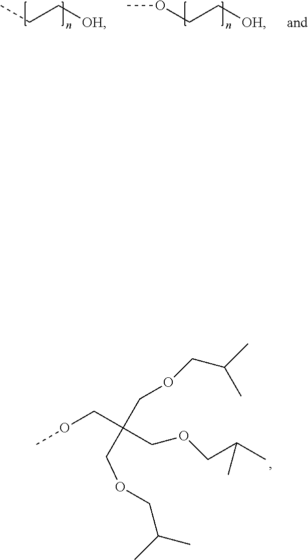

wherein n is 1-10.

In some embodiments of the compounds as disclosed herein, L is selected from the group consisting of:

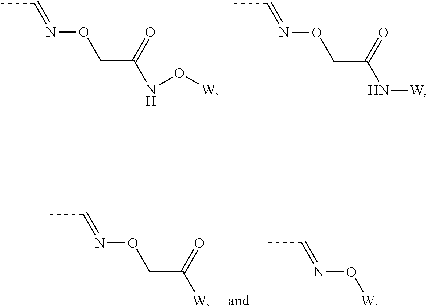

The presently-disclosed subject matter further includes methods of making the compounds as disclosed herein.

In some embodiments, a method is provided for making the compound

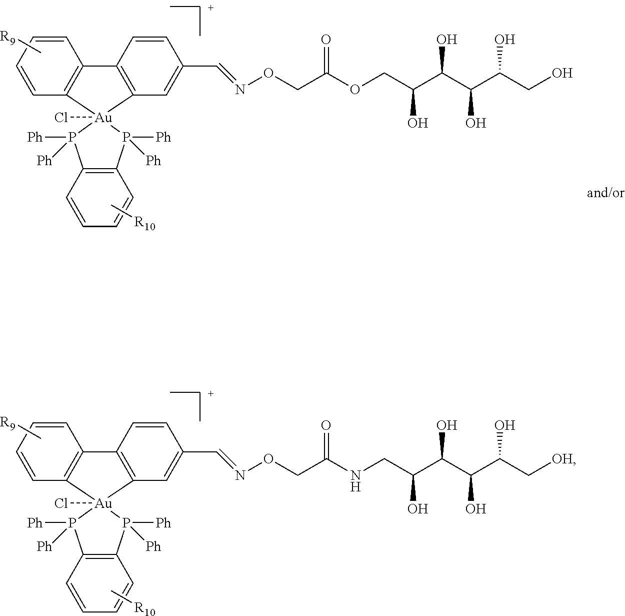

which comprises reacting

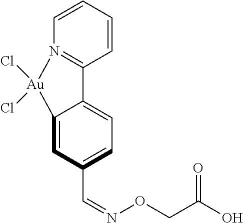

with sorbitol and/or glucamine in the presence of 1-Ethyl-3-(3-dimethylaminopropyl)carbodiimide hydrochloride (EDC), 1-hydroxybenzotriazole (HOBt), and anhydrous dimethylformamide (anh.DMF).

In some embodiments, the method further comprises obtaining the compound

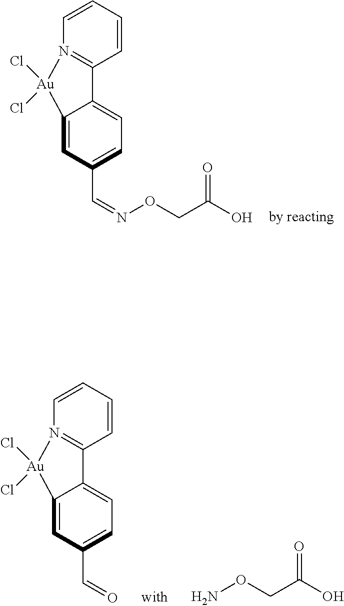

in the presence of HCL.

In some embodiments, the method further comprises obtaining the compound

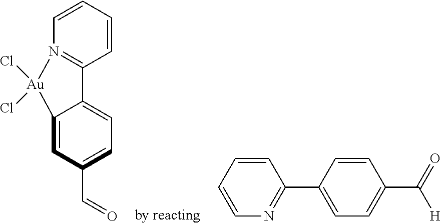

with HAuCl₄ in the presence of water.

The presently-disclosed subject matter further includes a pharmaceutical composition that includes a compound as disclosed herein and a pharmaceutically-acceptable carrier.

The presently-disclosed subject matter further includes a method of conferring anti-cancer activity to a cancer cell, which involves contacting a cancer cell with an effective amount of a compound or composition as disclosed herein. The cancer cell can be any type of cancer cell, with examples including, but not limited to leukemia, non-small cell lung cancer, colon cancer, central nervous system (CNS) cancer, melanoma, ovarian cancer, renal cancer, prostate cancer, or breast cancer.

In some embodiments, the effective amount is from about 10 nM to about 100 uM. In some embodiments, the cell is a cultured cell. In some embodiments, the cell is in a subject. In some aspects of the disclosed method, the subject has been diagnosed with a need for treatment of cancer.

In some embodiments, the conferring anti-cancer activity results in one or more of inhibiting proliferation of the cancer cell, inhibiting metastasis, and killing the cancer cell.

In some embodiments of the method, the cell is a cultured cell. In some embodiments of the method, the cell is in a subject. In some embodiments of the method, the subject is a mammal.

In some embodiments, the presently-disclosed subject matter is related to a method of increasing reactive oxygen species (ROS) in a cell, which involves contacting the cancer cell with an effective amount of one or more compounds or compositions as disclosed herein. In some embodiments, the effective amount is from about 10 nM to about 100 uM. In some embodiments, the cell is a cancer cell. In some embodiments, the cell is a cultured cell. In some embodiments, the cell is in a subject. In some embodiments, the subject is a mammal.

The presently-disclosed subject matter further includes use of a compound or composition as disclosed herein in a medicament for the treatment of a cancer.

The presently-disclosed subject matter further includes a method of modulating mitochondrial function in a cell, comprising: contacting a cell with an effective amount of a compound or composition as disclosed herein. In some embodiments of the method, the cell is a cancer cell. In some embodiments of the method, the cell is a cultured cell. In some embodiments of the method, the cell is in a subject. In some embodiments of the method, the subject is a mammal.

The presently-disclosed subject matter further includes use of a compound or composition as disclosed herein in a medicament for the treatment of a condition involving mitochondrial dysfunction.

The presently-disclosed subject matter further a method of increasing mitochondrial respiration in a cell, comprising contacting the cell with an effective amount of a compound or composition as disclosed herein. In some embodiments, the cell is an intestinal epithelial cell (IECs). In some embodiments, the cell is in a subject. In some embodiments, the subject has been diagnosed with inflammatory bowel disease (IBD). In some embodiments, the subject has been diagnosed with coronavirus induced enteritis. In some embodiments, the subject is a mammal. In some embodiments, the cell is a cultured cell. In some embodiments, the effective amount is between about 0.1 μM and about 10 μM.

The presently-disclosed subject matter further a method of modifying an intestinal microbiome of a subject in need thereof, which comprises administering to the subject a compound or composition as disclosed herein. In some embodiments, administration of the compound induces a shift to a healthy mixture of bacteria dominated by obligate anaerobes that produce short-chain fatty acids. In some embodiments, the subject is in need of treatment for one or more of dysbiotic microbiomes, intestinal inflammation, colitis, inflammatory bowel disease (IBD) and/or other metabolic and inflammatory autoimmune disorders, metabolic syndrome, cardiovascular disease and/or atherosclerotic heart disease, Alzheimer's disease, diabetes, autoimmune arthritis, autoimmune arthritides, fatty liver and/or a renal disease associated with end-stage renal failure.

While the terms used herein are believed to be well understood by those of ordinary skill in the art, certain definitions are set forth to facilitate explanation of the presently-disclosed subject matter.

Unless defined otherwise, all technical and scientific terms used herein have the same meaning as is commonly understood by one of skill in the art to which the invention(s) belong.

All patents, patent applications, published applications and publications, GenBank sequences, databases, websites and other published materials referred to throughout the entire disclosure herein, unless noted otherwise, are incorporated by reference in their entirety.

Where reference is made to a URL or other such identifier or address, it understood that such identifiers can change and particular information on the internet can come and go, but equivalent information can be found by searching the internet. Reference thereto evidences the availability and public dissemination of such information.

As used herein, the abbreviations for any protective groups, amino acids and other compounds, are, unless indicated otherwise, in accord with their common usage, recognized abbreviations, or the IUPAC-IUB Commission on Biochemical Nomenclature (See, iubmb.qmul.ac.uk/).

Although any methods, devices, and materials similar or equivalent to those described herein can be used in the practice or testing of the presently-disclosed subject matter, representative methods, devices, and materials are described herein.

The present application can “comprise” (open ended) or “consist essentially of” the components of the present invention as well as other ingredients or elements described herein. As used herein, “comprising” is open ended and means the elements recited, or their equivalent in structure or function, plus any other element or elements which are not recited. The terms “having” and “including” are also to be construed as open ended unless the context suggests otherwise.

Following long-standing patent law convention, the terms “a”, “an”, and “the” refer to “one or more” when used in this application, including the claims. Thus, for example, reference to “a cell” includes a plurality of such cells, and so forth.

Unless otherwise indicated, all numbers expressing quantities of ingredients, properties such as reaction conditions, and so forth used in the specification and claims are to be understood as being modified in all instances by the term “about”. Accordingly, unless indicated to the contrary, the numerical parameters set forth in this specification and claims are approximations that can vary depending upon the desired properties sought to be obtained by the presently-disclosed subject matter.

As used herein, the term “about,” when referring to a value or to an amount of mass, weight, time, volume, concentration or percentage is meant to encompass variations of in some embodiments±20%, in some embodiments±10%, in some embodiments±5%, in some embodiments±1%, in some embodiments±0.5%, in some embodiments±0.1%, in some embodiments±0.01%, and in some embodiments±0.001% from the specified amount, as such variations are appropriate to perform the disclosed method.

As used herein, ranges can be expressed as from “about” one particular value, and/or to “about” another particular value. It is also understood that there are a number of values disclosed herein, and that each value is also herein disclosed as “about” that particular value in addition to the value itself. For example, if the value “10” is disclosed, then “about 10” is also disclosed. It is also understood that each unit between two particular units are also disclosed. For example, if 10 and 15 are disclosed, then 11, 12, 13, and 14 are also disclosed.

As used herein, “optional” or “optionally” means that the subsequently described event or circumstance does or does not occur and that the description includes instances where said event or circumstance occurs and instances where it does not. For example, an optionally variant portion means that the portion is variant or non-variant.

In some aspects of the disclosed methods, the subject has been diagnosed with a need for treatment.

As used herein, the term “treatment” refers to the medical management of a patient with the intent to cure, ameliorate, stabilize, or prevent a disease, pathological condition, or disorder. This term includes active treatment, that is, treatment directed specifically toward the improvement of a disease, pathological condition, or disorder, and also includes causal treatment, that is, treatment directed toward removal of the cause of the associated disease, pathological condition, or disorder. In addition, this term includes palliative treatment, that is, treatment designed for the relief of symptoms rather than the curing of the disease, pathological condition, or disorder; preventative treatment, that is, treatment directed to minimizing or partially or completely inhibiting the development of the associated disease, pathological condition, or disorder; and supportive treatment, that is, treatment employed to supplement another specific therapy directed toward the improvement of the associated disease, pathological condition, or disorder.

As used herein, the term “prevent” or “preventing” refers to precluding, averting, obviating, forestalling, stopping, or hindering something from happening, especially by advance action. It is understood that where reduce, inhibit or prevent are used herein, unless specifically indicated otherwise, the use of the other two words is also expressly disclosed.

As used herein, the term “subject” refers to a target of administration or medical procedure. The subject of the herein disclosed methods can be a human or animal. The subject may also be a mammal. Thus, the subject of the herein disclosed methods can be a human, non-human primate, horse, pig, rabbit, dog, sheep, goat, cow, cat, guinea pig or rodent. The term does not denote a particular age or sex. Thus, adult and newborn subjects, as well as fetuses, whether male or female, are intended to be covered. A “patient” refers to a subject afflicted with a disease or disorder. The term “patient” includes human and veterinary subjects.

As used herein, the term “diagnosed” means having been subjected to a physical examination by a person of skill, for example, a physician, and found to have a condition that can be diagnosed or treated by the compounds, compositions, or methods disclosed herein. For example, “diagnosed with a disorder that creates intestinal mucosal injury” means having been subjected to a physical examination by a person of skill, for example, a physician, and found to have a condition that can be diagnosed or treated by a compound or composition that can promote healing of intestinal mucosal injury. Such a diagnosis can be in reference to a disorder, such as IBD or COVID induced enteritis, and the like, as discussed herein.

As used herein, the terms “administering” and “administration” refer to any method of providing a pharmaceutical preparation to a subject. Such methods are well known to those skilled in the art and include, but are not limited to, oral administration, transdermal administration, administration by inhalation, nasal administration, topical administration, intravaginal administration, ophthalmic administration, intraaural administration, intracerebral administration, rectal administration, and parenteral administration, including injectable such as intravenous administration, intra-arterial administration, intramuscular administration, and subcutaneous administration. Administration can be continuous or intermittent. In various aspects, a preparation can be administered therapeutically; that is, administered to treat an existing disease or condition. In further various aspects, a preparation can be administered prophylactically; that is, administered for prevention of a disease or condition.

As used herein, the term “effective amount” refers to an amount that is sufficient to achieve the desired result or to have an effect on an undesired condition. For example, a “therapeutically effective amount” refers to an amount that is sufficient to achieve the desired therapeutic result or to have an effect on undesired symptoms, but is generally insufficient to cause adverse side effects. The specific therapeutically effective dose level for any particular patient will depend upon a variety of factors including the disorder being treated and the severity of the disorder; the specific composition employed; the age, body weight, general health, sex and diet of the patient; the time of administration; the route of administration; the rate of excretion of the specific compound employed; the duration of the treatment; drugs used in combination or coincidental with the specific compound employed and like factors well known in the medical arts. For example, it is well within the skill of the art to start doses of a compound at levels lower than those required to achieve the desired therapeutic effect and to gradually increase the dosage until the desired effect is achieved. If desired, the effective daily dose can be divided into multiple doses for purposes of administration. Consequently, single dose compositions can contain such amounts or submultiples thereof to make up the daily dose. The dosage can be adjusted by the individual physician in the event of any contraindications. Dosage can vary, and can be administered in one or more dose administrations daily, for one or several days. Guidance can be found in the literature for appropriate dosages for given classes of pharmaceutical products. In further various aspects, a preparation can be administered in a “prophylactically effective amount”; that is, an amount effective for prevention of a disease or condition.

As used herein, the term “pharmaceutically acceptable carrier” refers to sterile aqueous or nonaqueous solutions, dispersions, suspensions or emulsions, as well as sterile powders for reconstitution into sterile injectable solutions or dispersions just prior to use. Examples of suitable aqueous and nonaqueous carriers, diluents, solvents or vehicles include water, ethanol, polyols (such as glycerol, propylene glycol, polyethylene glycol and the like), carboxymethylcellulose and suitable mixtures thereof, vegetable oils (such as olive oil) and injectable organic esters such as ethyl oleate. Proper fluidity can be maintained, for example, by the use of coating materials such as lecithin, by the maintenance of the required particle size in the case of dispersions and by the use of surfactants. These compositions can also contain adjuvants such as preservatives, wetting agents, emulsifying agents and dispersing agents. Prevention of the action of microorganisms can be ensured by the inclusion of various antibacterial and antifungal agents such as paraben, chlorobutanol, phenol, sorbic acid and the like. It can also be desirable to include isotonic agents such as sugars, sodium chloride and the like. Prolonged absorption of the injectable pharmaceutical form can be brought about by the inclusion of agents, such as aluminum monostearate and gelatin, which delay absorption. Injectable depot forms are made by forming microencapsule matrices of the drug in biodegradable polymers such as polylactide-polyglycolide, poly(orthoesters) and poly(anhydrides). Depending upon the ratio of drug to polymer and the nature of the particular polymer employed, the rate of drug release can be controlled. Depot injectable formulations are also prepared by entrapping the drug in liposomes or microemulsions which are compatible with body tissues. The injectable formulations can be sterilized, for example, by filtration through a bacterial-retaining filter or by incorporating sterilizing agents in the form of sterile solid compositions which can be dissolved or dispersed in sterile water or other sterile injectable media just prior to use. Suitable inert carriers can include sugars such as lactose. Desirably, at least 95% by weight of the particles of the active ingredient have an effective particle size in the range of 0.01 to 10 micrometers.

A residue of a chemical species, as used in the specification and concluding claims, refers to the moiety that is the resulting product of the chemical species in a particular reaction scheme or subsequent formulation or chemical product, regardless of whether the moiety is actually obtained from the chemical species. Thus, an ethylene glycol residue in a polyester refers to one or more —OCH₂CH₂O— units in the polyester, regardless of whether ethylene glycol was used to prepare the polyester. Similarly, a sebacic acid residue in a polyester refers to one or more —CO(CH₂)₈CO— moieties in the polyester, regardless of whether the residue is obtained by reacting sebacic acid or an ester thereof to obtain the polyester.

As used herein, the term “substituted” is contemplated to include all permissible substituents of organic compounds. In a broad aspect, the permissible substituents include acyclic and cyclic, branched and unbranched, carbocyclic and heterocyclic, and aromatic and nonaromatic substituents of organic compounds. Illustrative substituents include, for example, those described below. The permissible substituents can be one or more and the same or different for appropriate organic compounds. For purposes of this disclosure, the heteroatoms, such as nitrogen, can have hydrogen substituents and/or any permissible substituents of organic compounds described herein which satisfy the valences of the heteroatoms. This disclosure is not intended to be limited in any manner by the permissible substituents of organic compounds. Also, the terms “substitution” or “substituted with” include the implicit proviso that such substitution is in accordance with permitted valence of the substituted atom and the substituent, and that the substitution results in a stable compound, e.g., a compound that does not spontaneously undergo transformation such as by rearrangement, cyclization, elimination, etc.

In defining various terms, “A¹,” “A²,” “A³,” and “A⁴” are used herein as generic symbols to represent various specific substituents. These symbols can be any substituent, not limited to those disclosed herein, and when they are defined to be certain substituents in one instance, they can, in another instance, be defined as some other substituents.

The term “alkyl” as used herein is a branched or unbranched saturated hydrocarbon group of 1 to 24 carbon atoms, such as methyl, ethyl, n-propyl, isopropyl, n-butyl, isobutyl, s-butyl, t-butyl, n-pentyl, isopentyl, s-pentyl, neopentyl, hexyl, heptyl, octyl, nonyl, decyl, dodecyl, tetradecyl, hexadecyl, eicosyl, tetracosyl, and the like. The alkyl group can be cyclic or acyclic. The alkyl group can be branched or unbranched. The alkyl group can also be substituted or unsubstituted. For example, the alkyl group can be substituted with one or more groups including, but not limited to, optionally substituted alkyl, cycloalkyl, alkoxy, amino, ether, halide, hydroxy, nitro, silyl, sulfo-oxo, or thiol, as described herein. A “lower alkyl” group is an alkyl group containing from one to six carbon atoms.

Throughout the specification “alkyl” is generally used to refer to both unsubstituted alkyl groups and substituted alkyl groups; however, substituted alkyl groups are also specifically referred to herein by identifying the specific substituent(s) on the alkyl group. For example, the term “halogenated alkyl” specifically refers to an alkyl group that is substituted with one or more halide, e.g., fluorine, chlorine, bromine, or iodine. The term “alkoxyalkyl” specifically refers to an alkyl group that is substituted with one or more alkoxy groups, as described below. The term “alkylamino” specifically refers to an alkyl group that is substituted with one or more amino groups, as described below, and the like. When “alkyl” is used in one instance and a specific term such as “alkylalcohol” is used in another, it is not meant to imply that the term “alkyl” does not also refer to specific terms such as “alkylalcohol” and the like.

This practice is also used for other groups described herein. That is, while a term such as “cycloalkyl” refers to both unsubstituted and substituted cycloalkyl moieties, the substituted moieties can, in addition, be specifically identified herein; for example, a particular substituted cycloalkyl can be referred to as, e.g., an “alkylcycloalkyl.” Similarly, a substituted alkoxy can be specifically referred to as, e.g., a “halogenated alkoxy,” a particular substituted alkenyl can be, e.g., an “alkenylalcohol,” and the like. Again, the practice of using a general term, such as “cycloalkyl,” and a specific term, such as “alkylcycloalkyl,” is not meant to imply that the general term does not also include the specific term.

The term “cycloalkyl” as used herein is a non-aromatic carbon-based ring composed of at least three carbon atoms. Examples of cycloalkyl groups include, but are not limited to, cyclopropyl, cyclobutyl, cyclopentyl, cyclohexyl, norbornyl, and the like. The term “heterocycloalkyl” is a type of cycloalkyl group as defined above, and is included within the meaning of the term “cycloalkyl,” where at least one of the carbon atoms of the ring is replaced with a heteroatom such as, but not limited to, nitrogen, oxygen, sulfur, or phosphorus. The cycloalkyl group and heterocycloalkyl group can be substituted or unsubstituted. The cycloalkyl group and heterocycloalkyl group can be substituted with one or more groups including, but not limited to, optionally substituted alkyl, cycloalkyl, alkoxy, amino, ether, halide, hydroxy, nitro, silyl, sulfo-oxo, or thiol as described herein.

The term “polyalkylene group” as used herein is a group having two or more CH₂ groups linked to one another. The polyalkylene group can be represented by a formula —(CH₂)ₐ—, where “a” is an integer of from 2 to 500.

The terms “alkoxy” and “alkoxyl” as used herein to refer to an alkyl or cycloalkyl group bonded through an ether linkage; that is, an “alkoxy” group can be defined as —OA¹ where A¹ is alkyl or cycloalkyl as defined above. “Alkoxy” also includes polymers of alkoxy groups as just described; that is, an alkoxy can be a polyether such as —OA¹-OA² or —OA¹-(OA²)ₐ-OA³, where “a” is an integer of from 1 to 200 and A¹, A², and A³ are alkyl and/or cycloalkyl groups.

The term “alkenyl” as used herein is a hydrocarbon group of from 2 to 24 carbon atoms with a structural formula containing at least one carbon-carbon double bond. Asymmetric structures such as (A¹A²)C═C(A³A⁴) are intended to include both the E and Z isomers. This can be presumed in structural formulae herein wherein an asymmetric alkene is present, or it can be explicitly indicated by the bond symbol C═C. The alkenyl group can be substituted with one or more groups including, but not limited to, optionally substituted alkyl, cycloalkyl, alkoxy, alkenyl, cycloalkenyl, alkynyl, cycloalkynyl, aryl, heteroaryl, aldehyde, amino, carboxylic acid, ester, ether, halide, hydroxy, ketone, azide, nitro, silyl, sulfo-oxo, or thiol, as described herein.

The term “cycloalkenyl” as used herein is a non-aromatic carbon-based ring composed of at least three carbon atoms and containing at least one carbon-carbon double bound, i.e., C═C. Examples of cycloalkenyl groups include, but are not limited to, cyclopropenyl, cyclobutenyl, cyclopentenyl, cyclopentadienyl, cyclohexenyl, cyclohexadienyl, norbornenyl, and the like. The term “heterocycloalkenyl” is a type of cycloalkenyl group as defined above, and is included within the meaning of the term “cycloalkenyl,” where at least one of the carbon atoms of the ring is replaced with a heteroatom such as, but not limited to, nitrogen, oxygen, sulfur, or phosphorus. The cycloalkenyl group and heterocycloalkenyl group can be substituted or unsubstituted. The cycloalkenyl group and heterocycloalkenyl group can be substituted with one or more groups including, but not limited to, optionally substituted alkyl, cycloalkyl, alkoxy, alkenyl, cycloalkenyl, alkynyl, cycloalkynyl, aryl, heteroaryl, aldehyde, amino, carboxylic acid, ester, ether, halide, hydroxy, ketone, azide, nitro, silyl, sulfo-oxo, or thiol as described herein.

The term “alkynyl” as used herein is a hydrocarbon group of 2 to 24 carbon atoms with a structural formula containing at least one carbon-carbon triple bond. The alkynyl group can be unsubstituted or substituted with one or more groups including, but not limited to, optionally substituted alkyl, cycloalkyl, alkoxy, alkenyl, cycloalkenyl, alkynyl, cycloalkynyl, aryl, heteroaryl, aldehyde, amino, carboxylic acid, ester, ether, halide, hydroxy, ketone, azide, nitro, silyl, sulfo-oxo, or thiol, as described herein.

The term “cycloalkynyl” as used herein is a non-aromatic carbon-based ring composed of at least seven carbon atoms and containing at least one carbon-carbon triple bound. Examples of cycloalkynyl groups include, but are not limited to, cycloheptynyl, cyclooctynyl, cyclononynyl, and the like. The term “heterocycloalkynyl” is a type of cycloalkenyl group as defined above, and is included within the meaning of the term “cycloalkynyl,” where at least one of the carbon atoms of the ring is replaced with a heteroatom such as, but not limited to, nitrogen, oxygen, sulfur, or phosphorus. The cycloalkynyl group and heterocycloalkynyl group can be substituted or unsubstituted. The cycloalkynyl group and heterocycloalkynyl group can be substituted with one or more groups including, but not limited to, optionally substituted alkyl, cycloalkyl, alkoxy, alkenyl, cycloalkenyl, alkynyl, cycloalkynyl, aryl, heteroaryl, aldehyde, amino, carboxylic acid, ester, ether, halide, hydroxy, ketone, azide, nitro, silyl, sulfo-oxo, or thiol as described herein.

The term “aryl” as used herein is a group that contains any carbon-based aromatic group including, but not limited to, benzene, naphthalene, phenyl, biphenyl, phenoxybenzene, and the like. The term “aryl” also includes “heteroaryl,” which is defined as a group that contains an aromatic group that has at least one heteroatom incorporated within the ring of the aromatic group. Examples of heteroatoms include, but are not limited to, nitrogen, oxygen, sulfur, and phosphorus. Likewise, the term “non-heteroaryl,” which is also included in the term “aryl,” defines a group that contains an aromatic group that does not contain a heteroatom. The aryl group can be substituted or unsubstituted. The aryl group can be substituted with one or more groups including, but not limited to, optionally substituted alkyl, cycloalkyl, alkoxy, alkenyl, cycloalkenyl, alkynyl, cycloalkynyl, aryl, heteroaryl, aldehyde, amino, carboxylic acid, ester, ether, halide, hydroxy, ketone, azide, nitro, silyl, sulfo-oxo, or thiol as described herein. The term “biaryl” is a specific type of aryl group and is included in the definition of “aryl.” Biaryl refers to two aryl groups that are bound together via a fused ring structure, as in naphthalene, or are attached via one or more carbon-carbon bonds, as in biphenyl.

The term “aldehyde” as used herein is represented by a formula —C(O)H. Throughout this specification “C(O)” is a short hand notation for a carbonyl group, i.e., C═O.

The terms “amine” or “amino” as used herein are represented by a formula NA¹A²A³, where A¹, A², and A³ can be, independently, hydrogen or optionally substituted alkyl, cycloalkyl, alkenyl, cycloalkenyl, alkynyl, cycloalkynyl, aryl, or heteroaryl group as described herein.

The term “carboxylic acid” as used herein is represented by a formula —C(O)OH.

The term “ester” as used herein is represented by a formula —OC(O)A¹ or —C(O)OA¹, where A¹ can be an optionally substituted alkyl, cycloalkyl, alkenyl, cycloalkenyl, alkynyl, cycloalkynyl, aryl, or heteroaryl group as described herein. The term “polyester” as used herein is represented by a formula -(A¹O(O)C-A²-C(O)O)ₐ— or -(A¹O(O)C-A²-OC(O))ₐ—, where A¹ and A² can be, independently, an optionally substituted alkyl, cycloalkyl, alkenyl, cycloalkenyl, alkynyl, cycloalkynyl, aryl, or heteroaryl group described herein and “a” is an integer from 1 to 500. “Polyester” is as the term used to describe a group that is produced by the reaction between a compound having at least two carboxylic acid groups with a compound having at least two hydroxyl groups.

The term “ether” as used herein is represented by a formula A¹OA², where A¹ and A² can be, independently, an optionally substituted alkyl, cycloalkyl, alkenyl, cycloalkenyl, alkynyl, cycloalkynyl, aryl, or heteroaryl group described herein. The term “polyether” as used herein is represented by a formula -(A¹O-A²O)ₐ—, where A¹ and A² can be, independently, an optionally substituted alkyl, cycloalkyl, alkenyl, cycloalkenyl, alkynyl, cycloalkynyl, aryl, or heteroaryl group described herein and “a” is an integer of from 1 to 500. Examples of polyether groups include polyethylene oxide, polypropylene oxide, and polybutylene oxide.

The term “halide” as used herein refers to the halogens fluorine, chlorine, bromine, and iodine.

The term “heterocycle,” as used herein refers to single and multi-cyclic aromatic or non-aromatic ring systems in which at least one of the ring members is other than carbon. Heterocycle includes pyridine, pyrimidine, furan, thiophene, pyrrole, isoxazole, isothiazole, pyrazole, oxazole, thiazole, imidazole, oxazole, including, 1,2,3-oxadiazole, 1,2,5-oxadiazole and 1,3,4-oxadiazole, thiadiazole, including, 1,2,3-thiadiazole, 1,2,5-thiadiazole, and 1,3,4-thiadiazole, triazole, including, 1,2,3-triazole, 1,3,4-triazole, tetrazole, including 1,2,3,4-tetrazole and 1,2,4,5-tetrazole, pyridine, pyridazine, pyrimidine, pyrazine, triazine, including 1,2,4-triazine and 1,3,5-triazine, tetrazine, including 1,2,4,5-tetrazine, pyrrolidine, piperidine, piperazine, morpholine, azetidine, tetrahydropyran, tetrahydrofuran, dioxane, and the like.

The term “hydroxyl” as used herein is represented by a formula OH.

The term “ketone” as used herein is represented by a formula A¹C(O)A², where A¹ and A² can be, independently, an optionally substituted alkyl, cycloalkyl, alkenyl, cycloalkenyl, alkynyl, cycloalkynyl, aryl, or heteroaryl group as described herein.

The term “azide” as used herein is represented by a formula —N₃.

The term “nitro” as used herein is represented by a formula —NO₂.

The term “nitrile” as used herein is represented by a formula —CN.

The term “silyl” as used herein is represented by a formula —SiA¹A²A³, where A¹, A², and A³ can be, independently, hydrogen or an optionally substituted alkyl, cycloalkyl, alkoxy, alkenyl, cycloalkenyl, alkynyl, cycloalkynyl, aryl, or heteroaryl group as described herein.

The term “sulfo-oxo” as used herein is represented by a formulas —S(O)A¹, —S(O)₂A¹, —OS(O)₂A¹, or —OS(O)₂OA¹, where A¹ can be hydrogen or an optionally substituted alkyl, cycloalkyl, alkenyl, cycloalkenyl, alkynyl, cycloalkynyl, aryl, or heteroaryl group as described herein. Throughout this specification “S(O)” is a short hand notation for S═O. The term “sulfonyl” is used herein to refer to the sulfo-oxo group represented by a formula —S(O)₂A¹, where A¹ can be hydrogen or an optionally substituted alkyl, cycloalkyl, alkenyl, cycloalkenyl, alkynyl, cycloalkynyl, aryl, or heteroaryl group as described herein. The term “sulfone” as used herein is represented by a formula A¹S(O)₂A², where A¹ and A² can be, independently, an optionally substituted alkyl, cycloalkyl, alkenyl, cycloalkenyl, alkynyl, cycloalkynyl, aryl, or heteroaryl group as described herein. The term “sulfoxide” as used herein is represented by a formula A¹S(O)A², where A¹ and A² can be, independently, an optionally substituted alkyl, cycloalkyl, alkenyl, cycloalkenyl, alkynyl, cycloalkynyl, aryl, or heteroaryl group as described herein.

The term “thiol” as used herein is represented by a formula —SH.

The term “organic residue” defines a carbon containing residue, i.e., a residue comprising at least one carbon atom, and includes but is not limited to the carbon-containing groups, residues, or radicals defined hereinabove. Organic residues can contain various heteroatoms, or be bonded to another molecule through a heteroatom, including oxygen, nitrogen, sulfur, phosphorus, or the like. Examples of organic residues include but are not limited alkyl or substituted alkyls, alkoxy or substituted alkoxy, mono or di-substituted amino, amide groups, etc. Organic residues can preferably comprise 1 to 18 carbon atoms, 1 to 15, carbon atoms, 1 to 12 carbon atoms, 1 to 8 carbon atoms, 1 to 6 carbon atoms, or 1 to 4 carbon atoms. In a further aspect, an organic residue can comprise 2 to 18 carbon atoms, 2 to 15, carbon atoms, 2 to 12 carbon atoms, 2 to 8 carbon atoms, 2 to 4 carbon atoms, or 2 to 4 carbon atoms.

The term “pharmaceutically acceptable” describes a material that is not biologically or otherwise undesirable, i.e., without causing an unacceptable level of undesirable biological effects or interacting in a deleterious manner.

As used herein, the term “linker group” refers to a chemical moiety that covalently connects two distinct structural components of a compound, and can be selected to modulate spatial orientation, flexibility, electronic properties, solubility, or reactivity. As will be appreciated by one of ordinary skill in the art upon study of this document, representative linker groups include, but are not limited to, alkylene, arylene, heteroalkylene, heteroarylene, polyether chains (e.g., PEG), amide linkages, oxime ethers, carbamoyloximes, carboxyalkyloximes, and combinations thereof. The linker group can be linear, branched, cyclic, or polymeric, and can optionally include functional groups that participate in hydrogen bonding, ionization, or other interactions relevant to the compound's biological or chemical activity.

As used herein, the term “water soluble group” refers to a chemical moiety that, when incorporated into a compound, enhances the compound's solubility in water through ionization, hydrogen bonding, hydrophilic interactions, or other solubilizing mechanisms recognized in the art. Representative water soluble groups include, but are not limited to: ionizable acidic functional groups such as carboxylic acid and sulfonic acid; oxoacid amide derivatives such as sulfonamides and phosphoramidates; heterocyclic hydroxamic acids including isoxazole-OH, thiazole-OH, and oxazole-OH; cyclic imides and lactams; polyhydroxy alkyl groups such as sorbitol; amino polyols such as glucamine; simple sugars (e.g., monosaccharides); polymeric sugars (e.g., polysaccharides); linear and branched polyethers including polyethylene glycol (PEG), PEG ether derivatives, and PEG/PPG copolymers. As will be appreciated by one of ordinary skill in the art, such groups promote water solubility via mechanisms such as ion formation, hydrogen bonding, and polar interactions with aqueous media.

As used herein, the term “derivative” refers to a compound having a structure derived from the structure of a parent compound (e.g., a compounds disclosed herein) and whose structure is sufficiently similar to those disclosed herein and based upon that similarity, would be expected by one skilled in the art to exhibit the same or similar activities and utilities as the claimed compounds, or to induce, as a precursor, the same or similar activities and utilities as the claimed compounds. Exemplary derivatives include salts, esters, amides, salts of esters or amides, and N-oxides of a parent compound.

Compounds described herein can contain one or more double bonds and, thus, potentially give rise to cis/trans (E/Z) isomers, as well as other conformational isomers. Unless stated to the contrary, the invention includes all such possible isomers, as well as mixtures of such isomers.

Unless stated to the contrary, a formula with chemical bonds shown only as solid lines and not as wedges or dashed lines contemplates each possible isomer, e.g., each enantiomer and diastereomer, and a mixture of isomers, such as a racemic or scalemic mixture. Compounds described herein can contain one or more asymmetric centers and, thus, potentially give rise to diastereomers and optical isomers. Unless stated to the contrary, the present invention includes all such possible diastereomers as well as their racemic mixtures, their substantially pure resolved enantiomers, all possible geometric isomers, and pharmaceutically acceptable salts thereof. Mixtures of stereoisomers, as well as isolated specific stereoisomers, are also included. During the course of the synthetic procedures used to prepare such compounds, or in using racemization or epimerization procedures known to those skilled in the art, the products of such procedures can be a mixture of stereoisomers. Additionally, unless expressly described as “unsubstituted”, all substituents can be substituted or unsubstituted.

In some aspects, a structure of a compound can be represented by a formula:

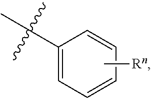

which is understood to be equivalent to a formula:

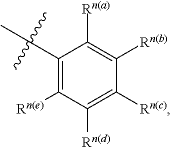

wherein n is typically an integer. That is, Rⁿ is understood to represent five independent substituents, R^{n(a)}, R^{n(b)}, R^{n(c)}, R^{n(d)}, R^{n(e)}. By “independent substituents,” it is meant that each R substituent can be independently defined. For example, if in one instance R^{n(a)} is alkyl, then R^{n(b)} is not necessarily alkyl in that instance. Likewise, when a group R is defined as four substituents, R is understood to represent four independent substituents, R^{a}, R^{b}, R^{c}, and R^{d}. Unless indicated to the contrary, the substituents are not limited to any particular order or arrangement.

Disclosed are the components to be used to prepare the compositions of the invention as well as the compositions themselves to be used within the methods disclosed herein. These and other materials are disclosed herein, and it is understood that when combinations, subsets, interactions, groups, etc. of these materials are disclosed that while specific reference of each various individual and collective combinations and permutation of these compounds cannot be explicitly disclosed, each is specifically contemplated and described herein. For example, if a particular compound is disclosed and discussed and a number of modifications that can be made to a number of molecules including the compounds are discussed, specifically contemplated is each and every combination and permutation of the compound and the modifications that are possible unless specifically indicated to the contrary. Thus, if a class of molecules A, B, and C are disclosed as well as a class of molecules D, E, and F and an example of a combination molecule, A-D is disclosed, then even if each is not individually recited each is individually and collectively contemplated meaning combinations, A-E, A-F, B-D, B-E, B-F, C-D, C-E, and C-F are considered disclosed. Likewise, any subset or combination of these is also disclosed. Thus, for example, the sub-group of A-E, B-F, and C-E would be considered disclosed. This concept applies to all aspects of this application including, but not limited to, steps in methods of making and using the compositions of the invention. Thus, if there are a variety of additional steps that can be performed it is understood that each of these additional steps can be performed with any specific aspect or combination of aspects of the methods of the invention.

The presently-disclosed subject matter is further illustrated by the following specific but non-limiting examples. The following examples may include compilations of data that are representative of data gathered at various times during the course of development and experimentation related to the present invention.

### EXAMPLES

### Example 1: Synthesis of Stable Gold-Based Molecules

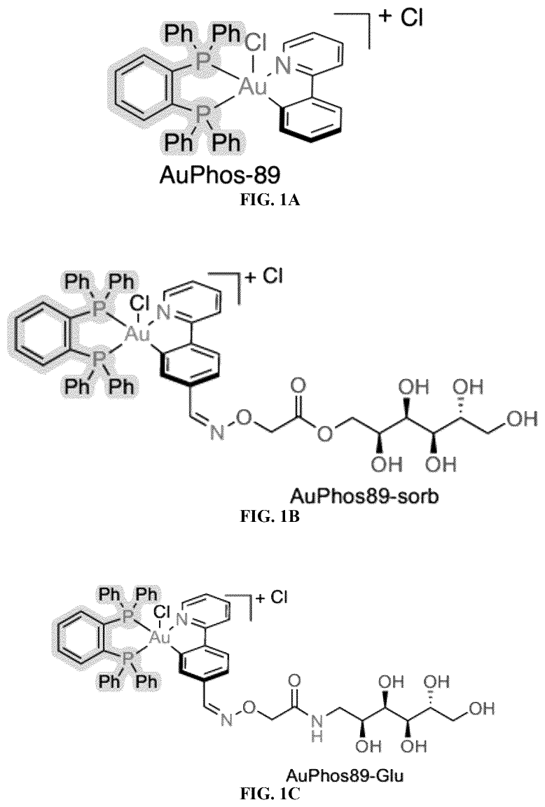

Leveraging the biocompatibility and redox adaptability of gold provides a unique approach to target mitochondria, a redox-sensitive organelle, and address undruggable cancer targets such as VDAC1, thereby opening new therapeutic avenues. A synthetic protocol was developed for C,N-cyclometalated ligands for improved stability in gold(III) anticancer agents (Scheme 1). Specifically, the compounds were synthesized via a facile C—H activation in water followed by ligand association steps. The compounds possess a rare geometry, a square bipyramid, for a gold(III) compound (FIG. 1A). These compounds overcome the rapid kinetic lability and thiol reactivity in physiological medium observed in previous gold compounds such as auranofin.

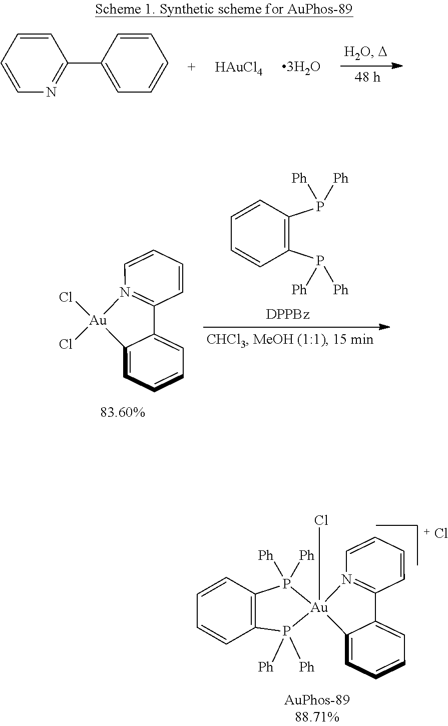

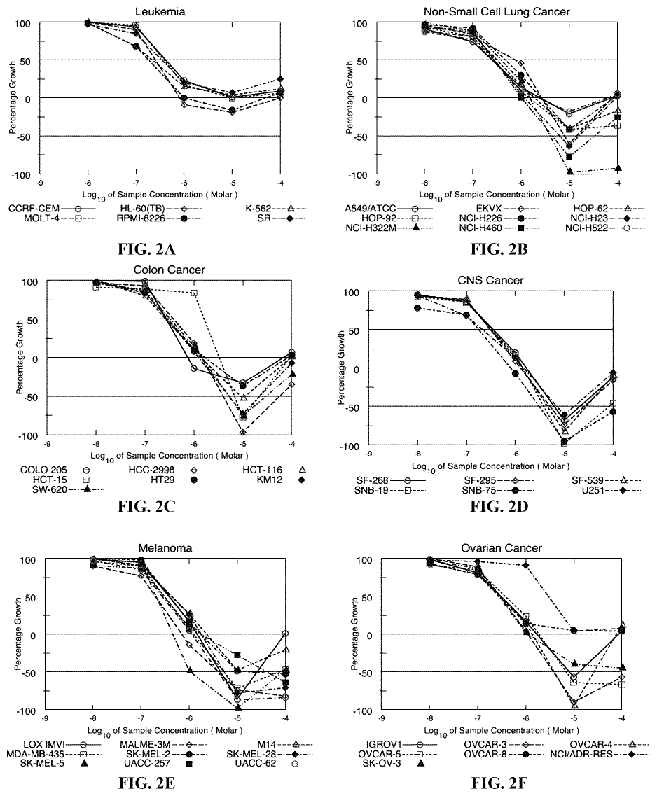

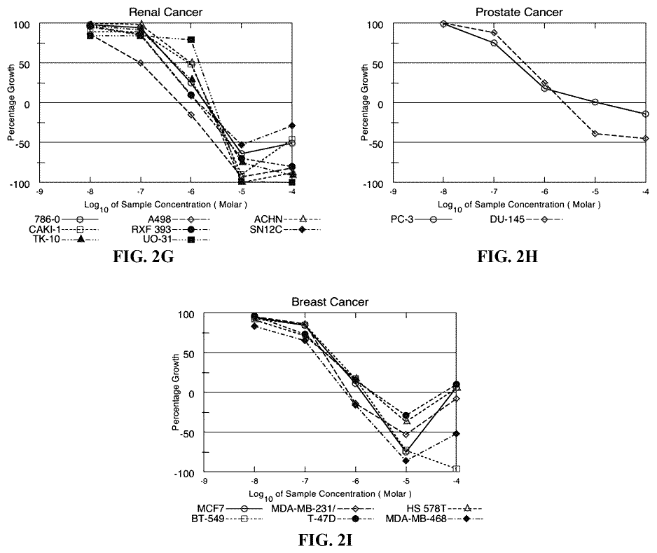

To understand the activity of the AuPhos (pronounced ‘goldphos’) scaffold, select compounds AuPhos-83, 84, and 89 were compared to a wide range of validated anticancer drugs via the well-established National Cancer Institute's reference panel screen set of 60 tumor cell types (NCI-60). In this screening service, the compound is administered in a single-dose of 10 μM to a range of 60 different cancer cell lines. Following a single-dose testing, the NCI selected all three compounds AuPhos-83, 84, and 89 for a 5-dose testing based on a threshold set by the NCI (accession codes: 7-NSC810362; 8-NSC810363; 9-NSC810364). The results show that the gold compounds are highly potent pan-cancer lethality with LD50 values of GI50 values within −150-700 nM. The 5-dose dose response summary graphs of AuPhos-89 (NSC810364) is shown in FIG. 2A-2I. AuPhos-89 was selected for further development due to relatively higher lethal potency across triple negative breast cancer (TNBC) panel, stability in reducing environment, and high synthetic yields >88%.

### Example 2: Identification of the Mitochondrial Protein VDAC1 as the Target of AuPhos-89

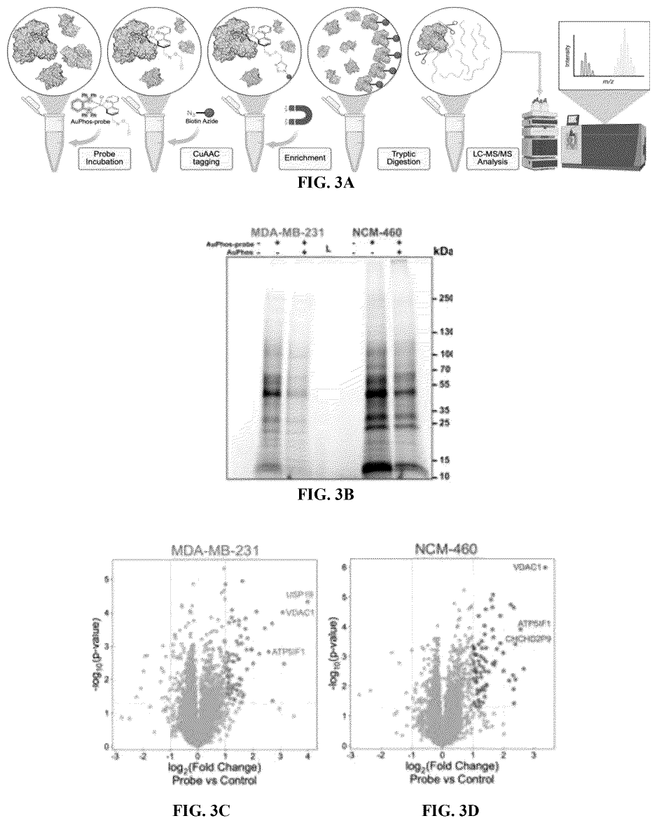

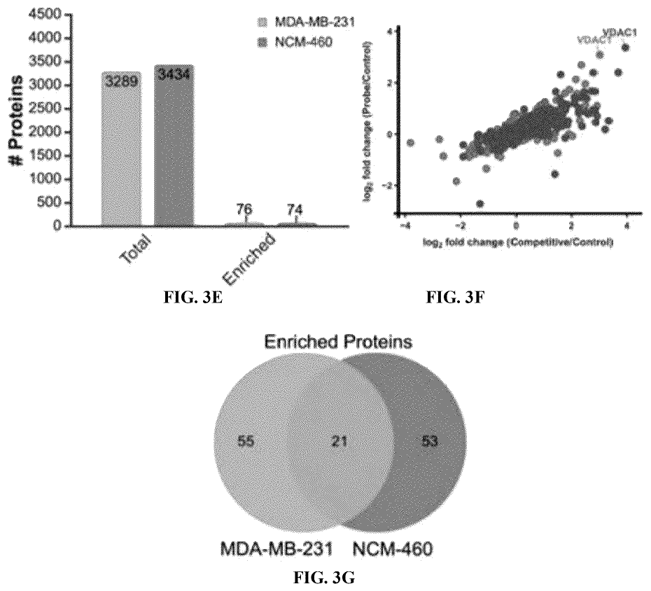

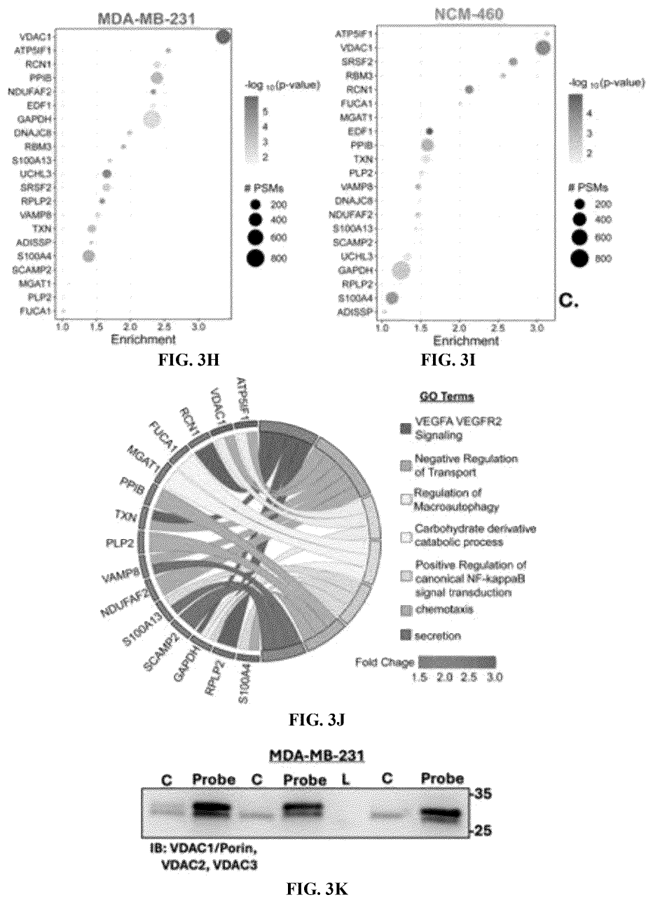

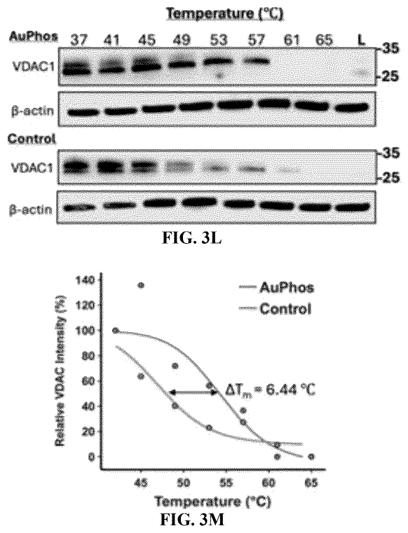

To identify the molecular target of AuPhos, AuPhos-alkyne chemical probe was synthesized for pull down studies enabled by azido-biotin labeling for proteomics in two different cell lysates (FIG. 3A). NCM-460 or MDA-MB-231 (for rigor) cell lysates were incubated with Au-Phos-alkyne at 10 μM for 2 h. CuAAC chemistry reaction was performed with azido rhodamine or biotin in lysates for 1 h and the lysates were incubated with streptavidin magnetic beads to pull down the biotinylated proteins. Lysates incubated with azido-rhodamine were run on SDS-PAGE for in-gel fluorescence (FIG. 3B). The enriched proteins with streptavidin beads from both treated or untreated cells were loaded onto SDS-PAGE and run for 5 min. The gel bands were cut and prepared for tryptic digestion and LC-MS/MS analysis of the enriched peptides to profile the targets. Proteins not found in the control group while showing a high abundance in the probe treatment group were identified as possible targets. Additionally, pretreatment of lysates with AuPhos served as a competition group to strengthen rigor of the target identification study. The volcano plot showed VDAC1 as the statistically significant, highly ranked, differentially expressed protein (FIG. 3C, 3D) in both cell lines used. With −70 enriched proteins out of over 3200 proteins identified (FIG. 3E, 3F), 21 overlapping enriched proteins were found from both MDA-MB-231 and NCM-460 lysates showing VDAC1 as significantly enriched (FIG. 3G, 3I). Mechanistic pathway analysis detailing gene ontology terms show molecular pathways in response to VDAC1 perturbation (FIG. 3J). Further validation by pull down immunoblotting (FIG. 3K) and cellular thermal shift assays (CETSA) with extrapolation (FIG. 3L, 3M) confirmed VDAC1 as the target of AuPhos.

### Example 3: VDAC1 is a Viable Cancer Target

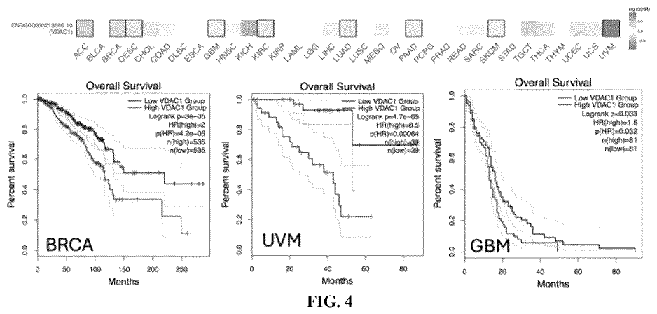

Targeting hub proteins represents a transformative strategy to overcome key cancer hallmarks, including metabolic reprogramming, therapy resistance, and uncontrolled cell proliferation. Among these, VDAC1 stands out as a mitochondrial gatekeeper with emerging therapeutic potential. VDAC1 is in the outer mitochondrial membrane (OMM), where it facilitates the transfer of critical metabolites, ions (including Ca²+), fatty acids, cholesterol, and reactive oxygen species (ROS) across the OMM, thus playing a central role in cellular metabolism and mitochondrial-mediated apoptosis. The VDAC protein family consists of three isoforms VDAC1, VDAC2, and VDAC3 with VDAC1 being the most abundant and functionally dominant. In aggressive and therapy-resistant tumors, VDAC1 is frequently overexpressed, correlating with tumor aggressiveness, resistance to therapy, and poor clinical outcomes (FIG. 4, data from TCGA). This makes VDAC1 not only a biomarker of cancer progression but also a promising therapeutic target. Compounds as disclosed herein are provided in connection with treating metabolically vulnerable tumors, including Hurthle cell carcinoma, melanoma, central nervous system (CNS) tumors, renal cancer, prostate cancer, and triple-negative breast cancer (TNBC).

### Example 4: AuPhos-89 Targeting of VDAC1 Disrupts Mitochondrial Function

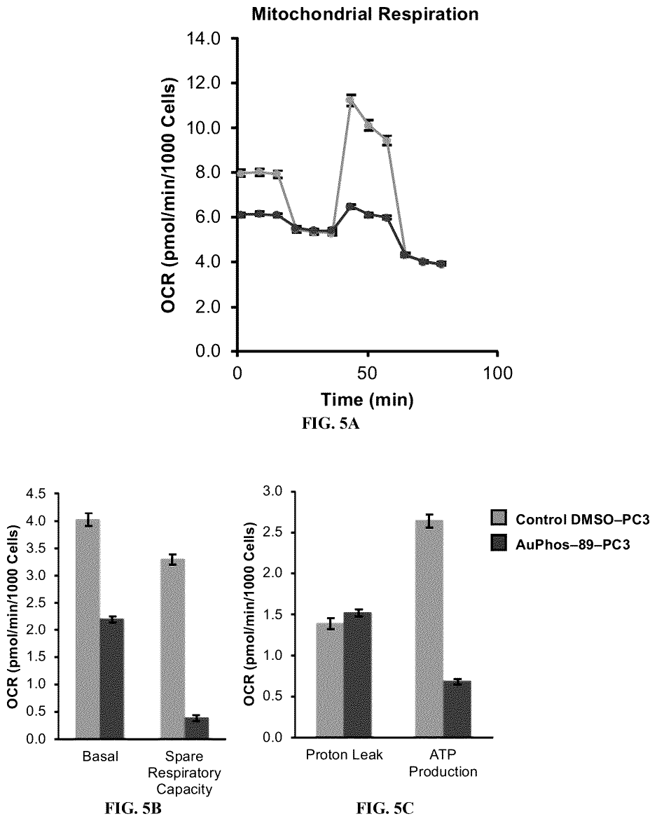

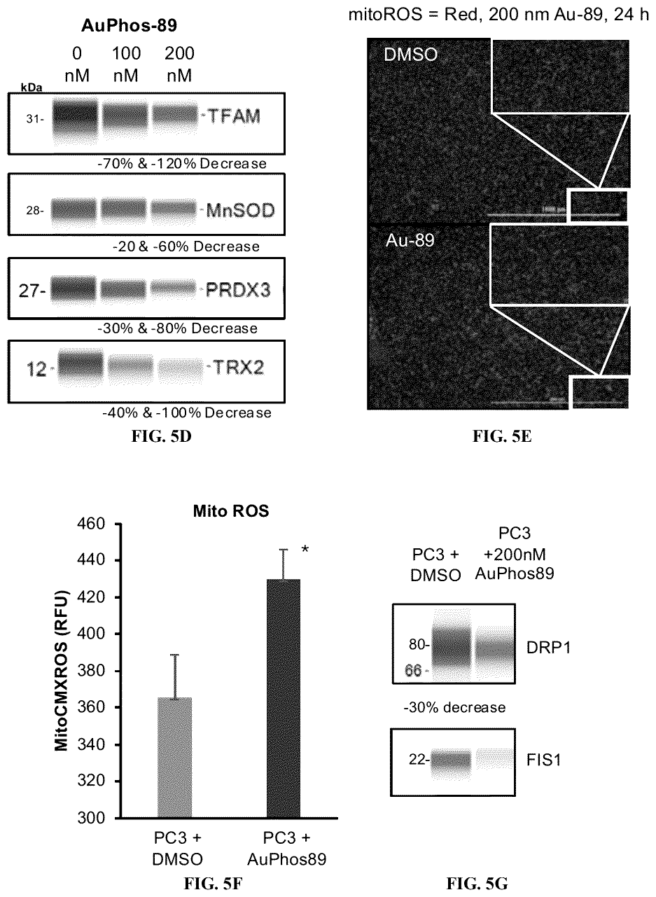

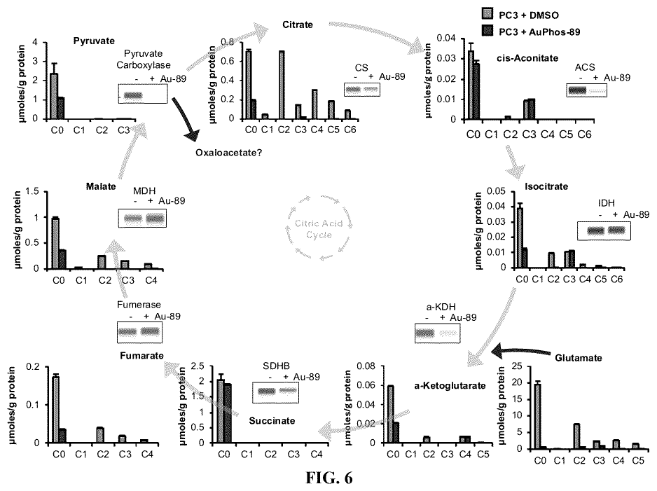

Mitochondrial function is significantly affected by AuPhos-89 targeting of the mitochondrial protein VDAC1. In PC3 prostate cancer cells, AuPhos-89 significantly induces mitochondrial dysfunction by repressing mitochondrial basal and maximum respiration leading to decreased spare respiratory capacity, increased proton leak, and decreased ATP energy production (FIG. 5A-C). Further, AuPhos-89 decreases the expression of mitochondrial biogenesis marker, TFAM, and antioxidant enzymes (FIG. 5D) with increased production of mitochondrial ROS (FIG. 5E, 5F) and depletion of mitochondrial fission proteins DRP1 and FIS1 (FIG. 5G). Additionally, using stable isotope resolved metabolomics (SIRM) and protein analysis in PC3 spheroids, metabolites (pyruvate, citrate, cis-aconitate, succinate) and associated proteins (pyruvate carboxylase, citrate synthase, aconitase, a-ketoglutarate dehydrogenase) of the TCA cycle were found to be significantly altered after exposure to AuPhos-89, indicative of OXPHOS inhibition (FIG. 6). These data support the hypothesis that AuPhos-89 has a unique and potent mitochondrial mechanism of action.

### Example 5: Preliminary AuPhos-89 Toxicology Studies Show the Potential for an Achievable Therapeutic Index

As per ICH S9 scientific guidelines for non-clinical evaluation for anti-cancer pharmaceuticals, preliminary safety studies were carried out using AuPhos-89. As seen in Table 1, AuPhos-89 is non-mutagenic, lipophilic, and has no effect on the liver and cardiac cells at concentrations higher than the therapeutic dose. Also, AuPhos-89 demonstrates stability in live cells and biological reductants such as L-GSH. Except for CYP3A4, all other CYP isoforms were not inhibited by AuPhos-89.

TABLE 1
In vitro ADMET showing non-clinical
evaluation for anti-cancer AuPhos-89

| Study Type | Outcome of Study |
|---|---|
| Bacterial reverse | Based on results of this study, it is concluded that |
| mutation test | AuPhos-89 is non-mutagenic to any tester strain of |
| (AMES) | Salmonella typhimurium, viz., TA98, TA100, in the |
| | presence or absence of S9 even at 350 mg/mL. |
| Lypophilicity | AuPhos-89 demonstrates lipophilicity and LgD > 2 |
| Hepatocellular | Cell viability in Hep2G cells show IC₅₀ > 1 μm. |
| Toxicity | |
| hERG Patch | Based on results of this study, it is concluded that |
| Claim Assay | AuPhos-89 showed weak inhibition of the hERG |
| | channel. The IC₅₀ of AuPhos-89 was determined |
| | to be >1 μm. |
| CYP Inhibition | Based on results of this study, it is concluded that |
| (IC₅₀) | AuPhos-89 had an IC₅₀ in the range of |
| | 4.8->25 μm for CYP1A2, 2B6, 2C8, 2C19, 2D6, 2C9. |
| | CYP3A4 had a much lower IC₅₋ value |
| | of 0.21 μm. |

### Example 6: AuPhos-89 Shows In Vivo Antitumor Activity

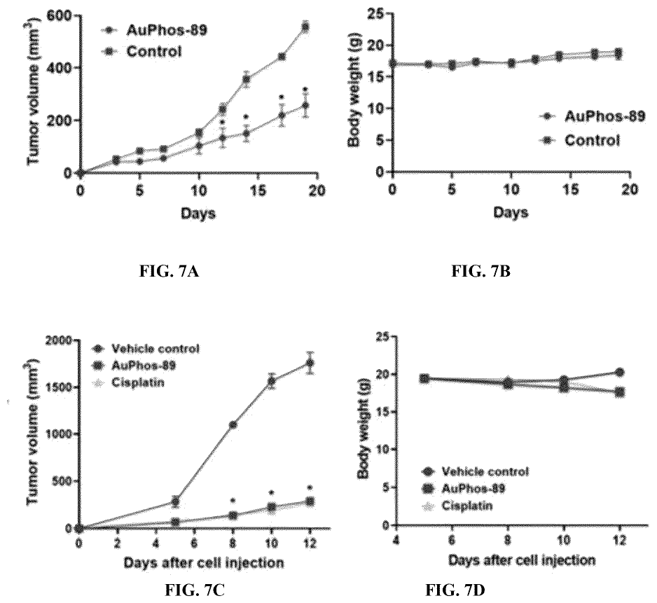

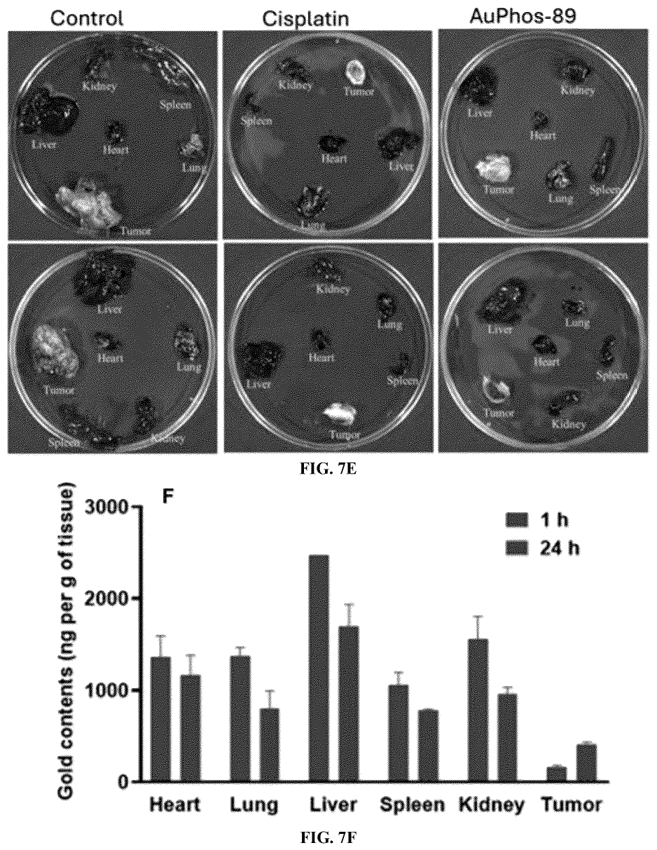

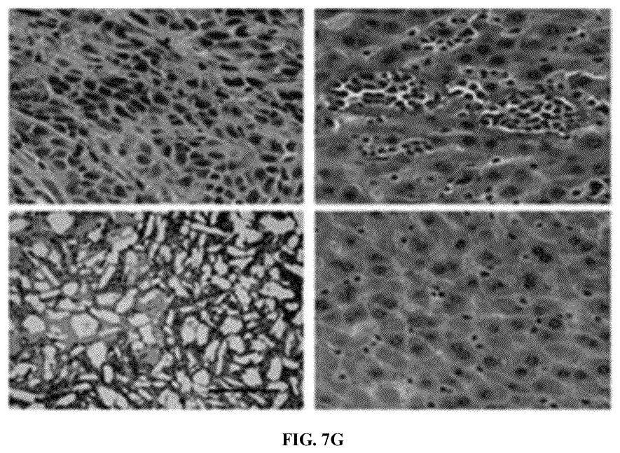

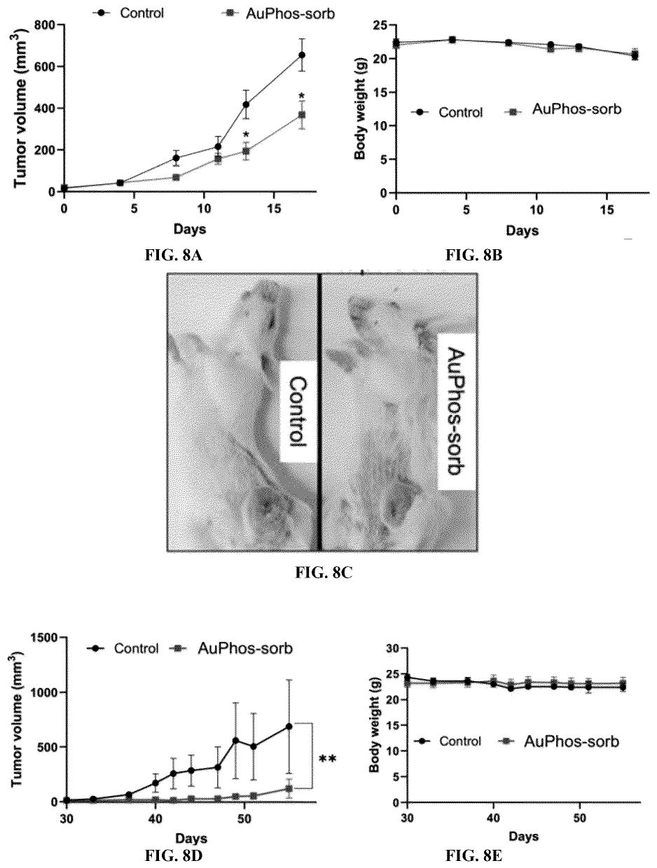

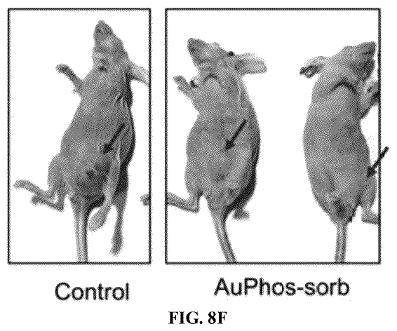

In vivo efficacy studies of AuPhos-89 in syngeneic 4T1 TNBC and AuPhos-89-sorb (FIG. 1B) in 4T1 and Sum159 xenografts showed significant in vivo anti-tumor efficacy using a schedule of administration that is tolerable (FIG. 7A-7G, FIG. 8A-8F).

### Example 7: Administration

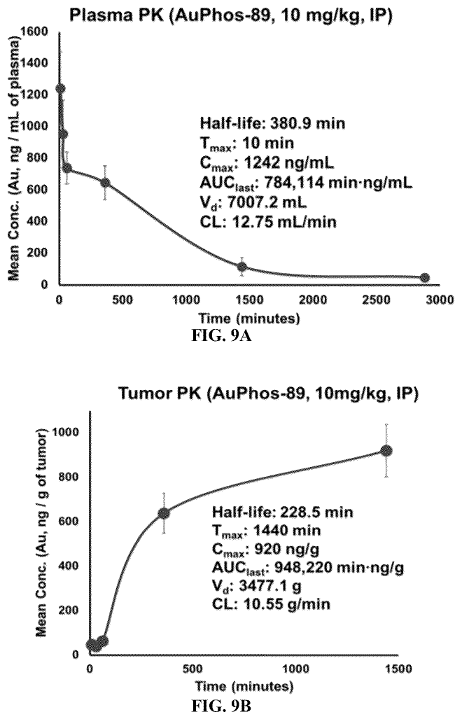

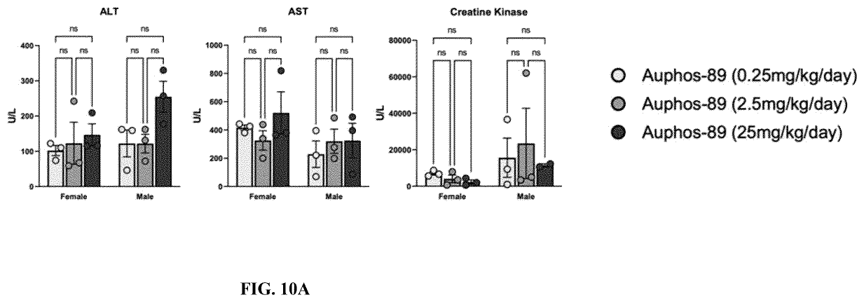

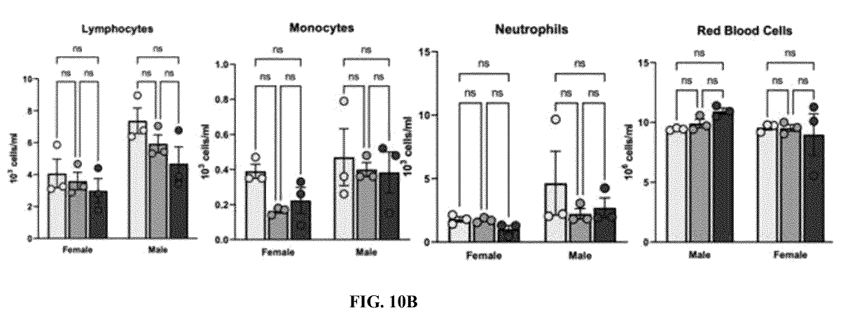

Based on the plasma and tumor pharmacokinetics and tissue distribution studies following intraperitoneal administration of parent AuPhos-89 in mice, a preliminary dosing schedule of 7-10 mg/kg 2×/week has been developed. Preliminary PK studies have shown that a single IP dose of AuPhos-89 reveals a half-life of ˜4 h, AUC 784,114 min ng/mL, Vd 7007.2 mL, and CL 12.75 mL/min (FIG. 9A-9B).

### Example 8: Water-Soluble Analogs of AuPhos

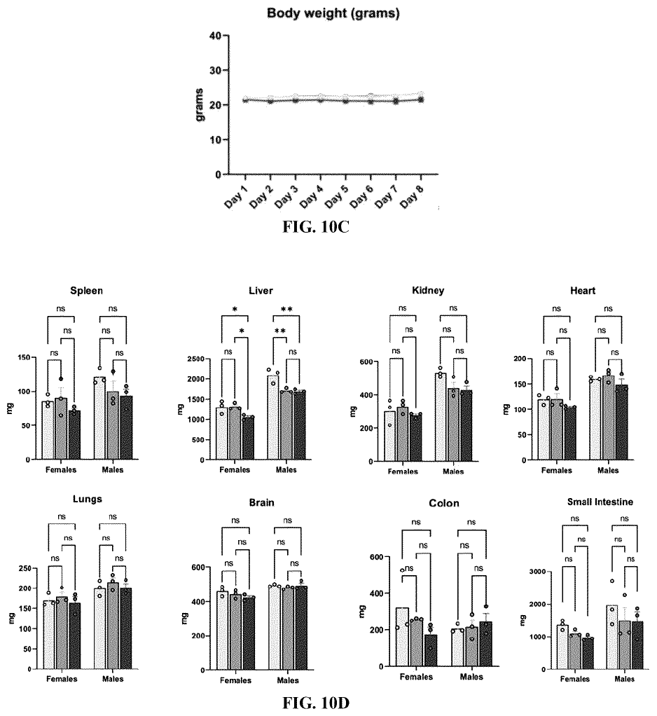

AuPhos-89 was modified to achieve water soluble derivatives (AuPhos-sorb (FIG. 1B) and AuPhos-Glu (FIG. 1C), Scheme 2) that do not require any complex formulation but maintains high potency and reduced toxicity. AuPhos-sorb was selected for further evaluation based on hydrolysable sorbitol fragment via the ester bond by intracellular esterases. A preliminary 7-day toxicity study in mice showed a good safety profile at 25 mg/kg PO dose as demonstrated by no overt lethargy or loss in body weight, unaltered blood chemistry and liver enzymes, and blood cell counts, indicating that a therapeutic index is achievable with a VDAC1-targeting agent (FIG. 10A-10D).

AuPhos-sorb targets VDAC. Target identification and validation studies confirmed engagement with the mitochondrial VDAC. AuPhos-sorb inhibits cancer metastasis. Both in vitro and in vivo studies using metastatic models of cancer demonstrate inhibition of cell invasion, migration, and tumor metastasis. AuPhos-sorb inhibits tumor growth. Different models of cancer were used to study efficacy of AuPhos-sorb and profound tumor growth inhibition was found.

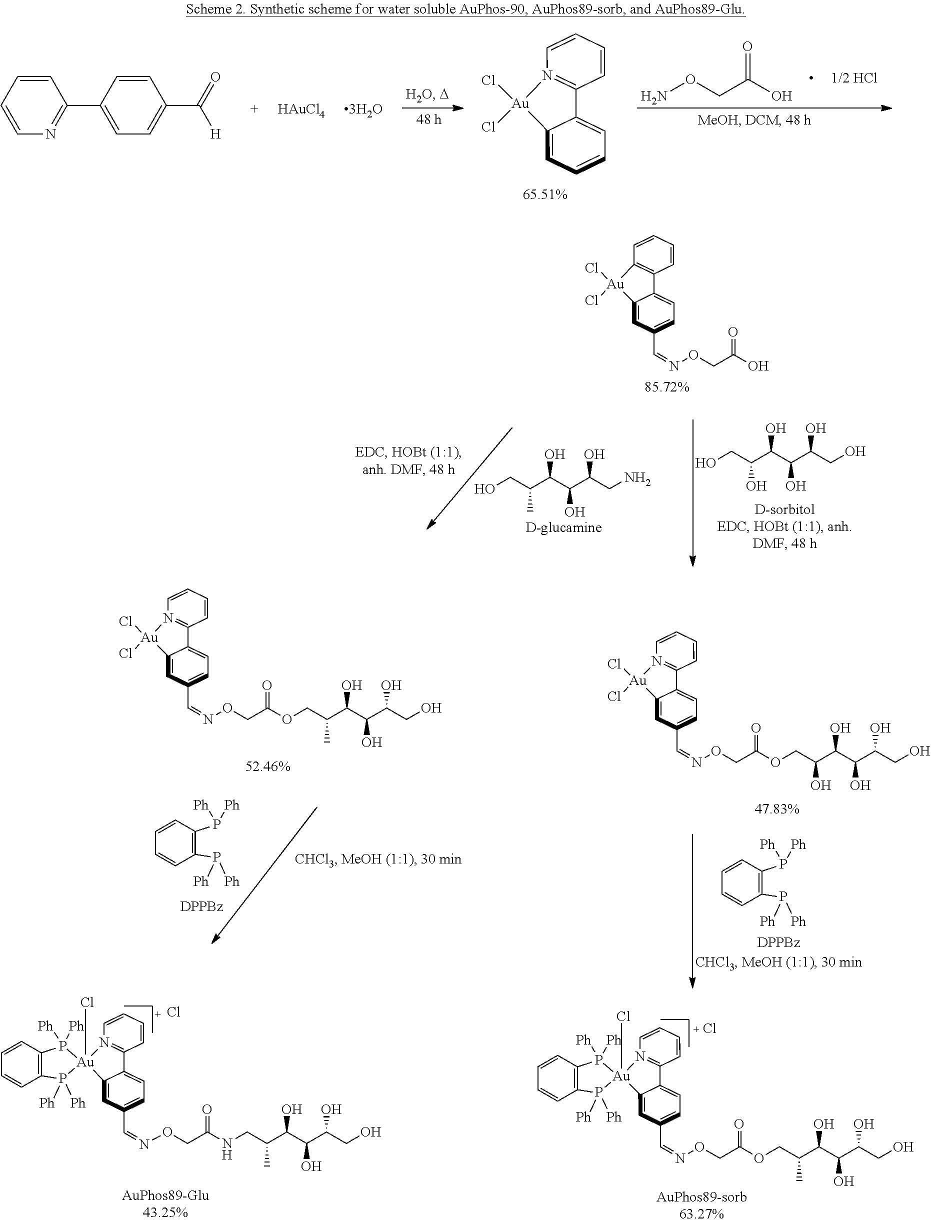

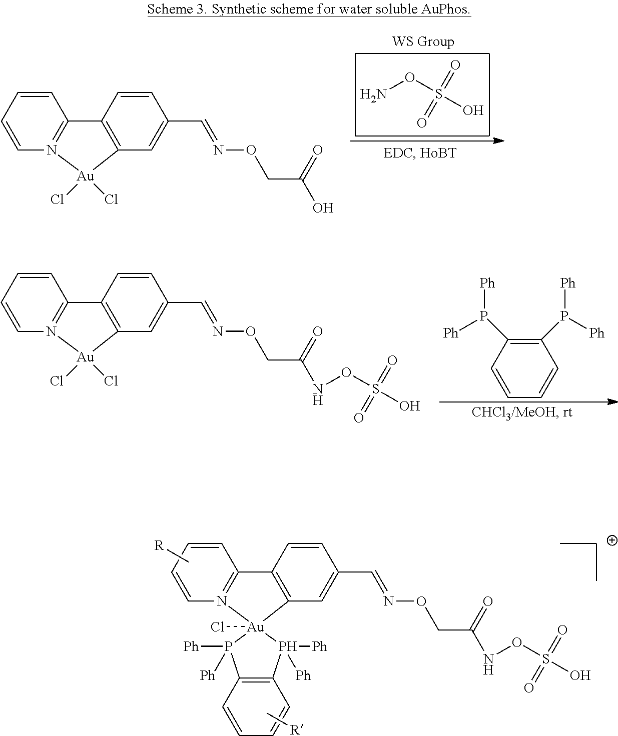

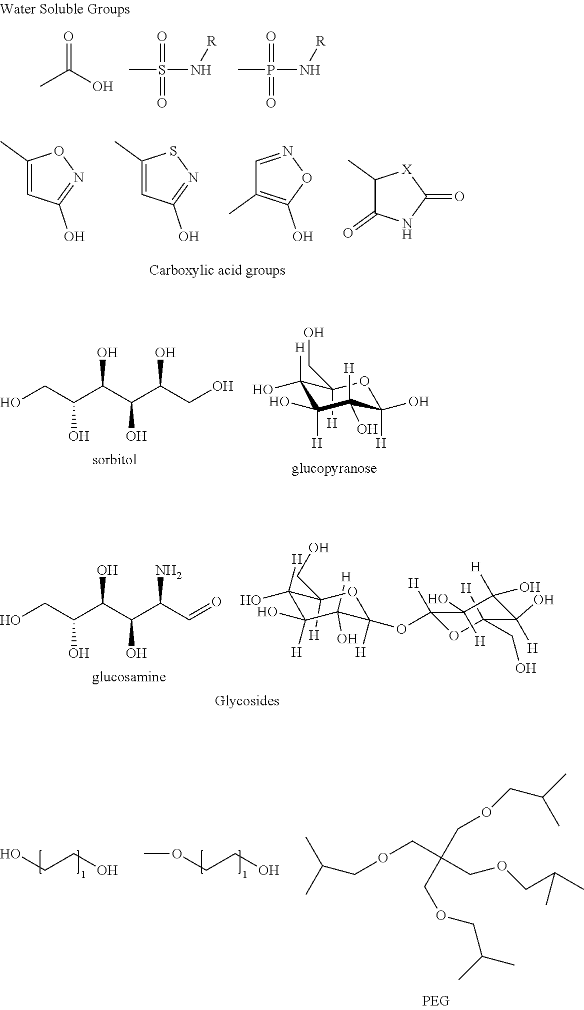

### Synthesis of Dichloro(4-(2-pyridyl)benzaldehyde)gold(111)

In a 30 mL pressure vessel, 4-(2-pyridyl)benzaldehyde (0.1782 g, 0.9727 mmol) and HAuCl₄·3H₂O (0.3819 g, 0.9697 mmol) were dissolved in distilled water (20 mL). The reaction mixture was stirred for 48 hours at 130° C. The precipitate was then vacuum filtered and washed with DI H₂O, EtOH, and diethyl ether (3×5 mL each) to afford an off-white solid (0.2859 g, 65.51% yield). ¹H NMR (400 MHz, DMSO) δ 10.02 (s, 1H), 9.57 (dd, J=6.1, 1.5 Hz, 1H), 8.54 (dd, J=8.1, 1.6 Hz, 1H), 8.47 (td, J=7.7, 1.5 Hz, 1H), 8.30 (d, J=1.5 Hz, 1H), 8.23 (d, J=7.9 Hz, 1H), 8.01 (dd, J=7.9, 1.5 Hz, 1H), 7.88 (ddd, J=7.6, 6.0, 1.6 Hz, 1H).

### Synthesis of Dichloro(4-(2-pyridyl)CH₃COOH-oxime)gold(III)

In a 50 mL round bottom flask, dichloro(4-(2-pyridyl)benzaldehyde)gold(III) (0.6186 g, 1.3745 mmol) and carboxymethoxylamine hemihydrochloride (0.4506 g, 4.133 mmol) were suspended in 20 mL of DCM:MeOH (1:1). The reaction mixture was stirred for 48 hours at room temperature, after which the mixture was centrifuged at 5,000 RPMs for 10 minutes at room temperature. The resulting pellet was resuspended in 20 mL MeOH and recentrifuged with 20 mL of MeOH (2 times) to afford a white solid (0.6163 g, 85.72% yield). ¹H NMR (400 MHz, DMSO) δ 12.91 (s, 1H), 9.55-9.46 (m, 1H), 8.44-8.35 (m, 3H), 8.04-7.96 (m, 2H), 7.78 (td, J=5.9, 3.4 Hz, 1H), 7.64 (dd, J=8.0, 1.5 Hz, 1H), 4.72 (s, 2H).

### Synthesis of Dichloro(4-(2-pyridyl)-D-glucamine-oxime)gold(III)

To a 100 mL Schlenk flask fitted with magnetic stir bar, dichloro(4-(2-pyridyl)CH₃COOH-oxime)gold(III) (0.7760 g, 1.483 mmol), D-glucamine (0.2720 g, 1.501 mmol), hydroxybenzotriazole (0.2019 g, 1.494 mmol), and 1-ethyl-3-(3-dimethylaminopropyl)carbodiimide (0.2910 g, 1.510 mmol) were added. The flask was purged and backfilled with nitrogen followed by the addition of anhydrous DMF (5 mL) and DCM (5 mL). The reaction was stirred for two days at room temperature and then quenched with water. The resulting suspension was centrifuged for 10 minutes at 15° C. three times, decanting supernatant, and resuspending in 30 mL DI H₂O each time. The resulting pellet was dried overnight with a stream of N₂ to afford a brown solid. Boiling methanol was added to the solid, and further filtered to obtain a yellow filtrate. The filtrate was evaporated, via rotavap to obtain a yellow solid (0.1176 g, 11.55% yield).

### Synthesis of (2-(p-tolyl)pyridine-D-glucamine-oxime)[1,2-Bis(diphenylphosphino)benzene]gold(III)

To a 25 mL round bottom flask, dichloro(4-(2-pyridyl)-D-glucamine-oxime)gold(III) (0.8702 g, 1.268 mmol) and 1,2-Bis(diphenylphosphino)benzene (0.5690 g, 1.275 mmol) were suspended in 10 mL CHCl₃ and 5 mL MeOH. The reaction mixture was stirred for 35 minutes at room temperature. The final compound was obtained via separation with flash chromatography using CombiFlashR Rf⁺ Lumen with 15:85/MeOH:CH₂Cl₂. Yield: 466.8 mg, 34.51%. ¹H NMR (400 MHz, DMSO)¹H NMR (400 MHz, DMSO) δ 8.27 (td, J=8.6, 3.6 Hz, 1H), 8.17 (s, 1H), 8.10-7.66 (m, 20H), 7.63-7.44 (m, 5H), 6.84 (dd, J=7.5, 5.0 Hz, 1H), 6.35 (s, 1H), 4.96-4.81 (m, 1H), 4.59 (s, 3H), 4.44 (d, J=17.3 Hz, 3H), 4.13 (s, 1H), 3.73-3.54 (m, 4H), 3.17 (s, 3H).

### Synthesis of (2-(p-tolyl)pyridine-PEG-oxime)gold(III)

To a 100 mL Schlenk flask fitted with a magnetic stir bar, dichloro(4-(2-pyridyl)CH₃COOH-oxime)gold(III)(0.231 g, 0.442 mmol), polyethylene glycol (0.248 g, 0.442), 1-ethyl-3-(3-dimethylaminopropyl)carbodiimide (0.254 g, 1.325 mmol), and 4-(dimethylamino)pyridine (0.162 g, 1.325 mmol) were added. The flask was purged and backfilled with nitrogen on ice, followed by the addition of anhydrous DMF (2 mL). The reaction was stirred for 5 minutes while on ice, then allowed to reach room temperature and stirred at 40° C. After one day, the reaction solution was transferred to a separatory funnel where it was acidified to a pH between 1 and 3 with 1M HCl. Workup with DCM/brine yielded crude product that was further purified with flash chromatography.

General Amide Coupling of D-Sorbitol, D-Glucamine and Hydroxylamine-O-Sulfonic Acid to Dichloro(4-(2-pyridyl)CH₃COOH-oxime)gold(III):

To a 100 mL Schlenk flask fitted with magnetic stir bar, dichloro(4-(2-pyridyl)CH₃COOH-oxime)gold(III), selected amine, hydroxybenzotriazole, and 1-ethyl-3-(3-dimethylaminopropyl)carbodiimide were added at equivalent molar amounts. The flask was purged and backfilled with nitrogen followed by the addition of anhydrous DMF (5 mL). The reaction was stirred for two days at room temperature and quenched with water. The resulting suspension was centrifuged for 10 minutes at 15° C. three times, decanting supernatant, and resuspending in 30 mL DI H₂O each time. The resulting pellet was dried overnight with a stream of N₂ to afford a solid.

### General DPPBz Coupling for Final WS-AuPhos Compounds:

To a 25 mL round bottom flask, water-soluble[C\hat{ }N]cyclometalated Au(III) and 1,2-Bis(diphenylphosphino)benzene were suspended in 10 mL CHCl₃ at equivalent molar ratio. The reaction mixture was stirred for 30 minutes at room temperature. The final compound was obtained via separation with flash chromatography using CombiFlashR Rf⁺ Lumen with 10:90/MeOH:CH₂Cl₂.

### Example 9: AuPhos Compounds for Colitis, Including Ulcerative Colitis (UC), Crohn's Disease (CD), and Inflammatory Bowel Disease (IBD)

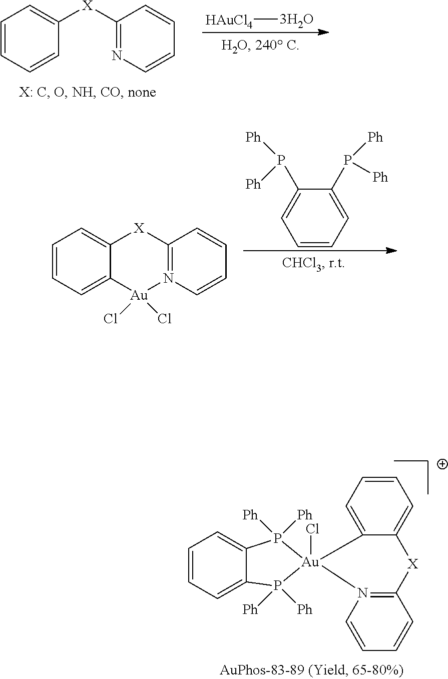

AuPhos enhances mitochondrial function in Intestinal Epithelial Cells (IECs). AuPhos was tested for its ability to enhance mitochondrial respiration in intestinal epithelial cells. The studies demonstrated that AuPhos increases the levels of mitochondrial complexes I through V, raises the oxygen consumption rate, and induces hypoxia in IECs. Additionally, AuPhos upregulates Nrf2 and PGC1α, which promotes mitochondrial biogenesis. No genotoxicity was observed in Ames tests. (Data not shown).

AuPhos improves colitis outcomes in mouse models. AuPhos was administered to mice with DSS-induced colitis and to piroxicam-accelerated IL10 knockout mice. AuPhos treatment significantly reduced weight loss, disease activity index, and histological colitis scores in these models. The compound improved mucosal healing, enhanced barrier function, and lowered proinflammatory cytokine mRNA levels. In chronic colitis models, AuPhos also decreased disease activity index, fecal LCN2, and histological severity, while increasing mitochondrial complex mRNA. (Data not shown).

AuPhos modulates gut microbiota composition. The fecal microbiota of wild-type, DSS colitis, and IL10 knockout mice treated with AuPhos were analyzed. AuPhos reduced the relative abundance of Proteobacteria, which are facultative anaerobic Enterobacteriaceae, and increased the presence of obligate anaerobic bacteria such as Firmicutes and Clostridia. In humanized IL10 knockout mice, AuPhos promoted a shift in the microbiome toward healthy anaerobes, resembling the profiles of donor IBD patients. (Data not shown).

AuPhos promotes ulcer healing and crypt branching in human colonoids. The effects of AuPhos were tested on human colonoids derived from areas of active and inactive colitis. Colonoids from active colitis exhibited reduced crypt branching and lower levels of mitochondrial complexes. Treatment with AuPhos increased the mRNA expression of mitochondrial complex IV (COX5B) and complex V (ATP5A), and restored crypt branching in mitochondria-deficient colonoids. (Data not shown).

AuPhos improves barrier function in colitis models. FITC-dextran permeability and trans-epithelial electrical resistance assays were performed in DSS colitis mice and human colonoid monolayers. AuPhos treatment improved barrier function, reduced epithelial permeability, and increased TEER in TNF-treated human colonoid monolayers. (Data not shown).

AuPhos reduces colitis severity and corrects dysbiosis in humanized mouse models. AuPhos was tested in germ-free IL10 knockout mice reconstituted with human IBD stool. AuPhos treatment reduced colitis severity, as measured by disease activity index, histology, and fecal LCN2. It also increased tissue hypoxia, improved mitochondrial function, and shifted the microbiome toward obligate anaerobes, resulting in reduced Proteobacteria. (Data not shown).

Pharmacokinetic and toxicity studies were conducted in mice to determine the absorption, tissue distribution, maximum tolerated dose, and toxicokinetics of AuPhos. The compound showed limited systemic absorption (less than 10%), with a predicted maximum tolerated dose of 250 mg/kg. Toxicity and tissue distribution were assessed. (Data not shown).

AuPhos targets DNA-PKcs and promotes mitochondrial biogenesis. Chemoproteomic and biophysical studies were performed to identify molecular targets of AuPhos. The results showed that AuPhos binds DNA-PKcs, cytoplasmic dynein, and other proteins involved in mitochondrial quality and biogenesis. Inhibition of DNA-PKcs by AuPhos was found to enhance mitochondrial biogenesis via activation of PGC1α. (Data not shown).

### Example 10: AuPhos-Glucamine Enhances Mitochondrial Biogenesis and Antioxidant Effects in IBD

AuPhos-glucamine was prepared by adding a glucamine group to the aryl pyridine ring of the parent AuPhos molecule. The water-soluble AuPhos-glucamine was tested for its effects on mitochondrial respiration, biogenesis, and antioxidant enzyme levels in human IBD tissue and cell models. Treatment with water-soluble AuPhos-glucamine increased the oxygen consumption rate by 38% in murine intestinal epithelial cells and elevated mitochondrial complex and biogenesis proteins in human colonic biopsies and NCM460 cells. The compound also increased mitochondrial mass and upregulated antioxidant markers such as MnSOD, glutathione peroxidase, thioredoxin reductase, and catalase. Furthermore, water-soluble AuPhos-glucamine significantly increased mRNA levels for mitochondrial complex genes and antioxidant enzymes in both control and TNF-treated samples. These findings indicate that water-soluble AuPhos-glucamine promotes mitochondrial biogenesis, elevates mitochondrial complex levels, and enhances antioxidant enzyme expression in human colonic tissue, suggesting that it may enhance mucosal repair in IBD by reducing cellular reactive oxygen species and improving cytoprotection. (Data not shown).

All publications, patents, and patent applications mentioned in this specification are herein incorporated by reference to the same extent as if each individual publication, patent, or patent application was specifically and individually indicated to be incorporated by reference, including the references set forth in the following list:

### REFERENCES

1. International Patent Application Publication No. WO 2022/026753.
2. International Patent Application Publication No. WO 2022/026757.
3. International Patent Application Publication No. WO 2022/072545.

It will be understood that various details of the presently disclosed subject matter can be changed without departing from the scope of the subject matter disclosed herein. Furthermore, the foregoing description is for the purpose of illustration only, and not for the purpose of limitation.

What is claimed is:

1. A compound of the formula: 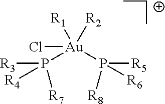
wherein R₁ and R₂, taken together with the gold (Au) to which they are bound, form a polycyclic moiety selected from the group consisting of: 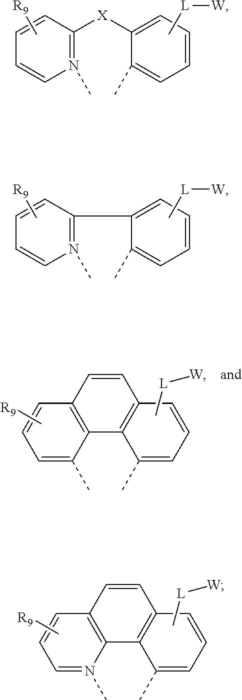
X is selected from the group consisting of CH₂, O, NH, C═O, C═NO-alkynyl, S, or aryl;
R₃, R₄, R₅, R₆ are each independently selected from the group consisting of H, alkyl, and aryl, or R₃ and R₄ and/or R₅ and R₆, taken together with the phosphorus (P) to which they are bound, form a cyclic moiety;
R₇ and R₈, together with the phosphorus (P) to which they are bound, form a structure selected from the group consisting of: 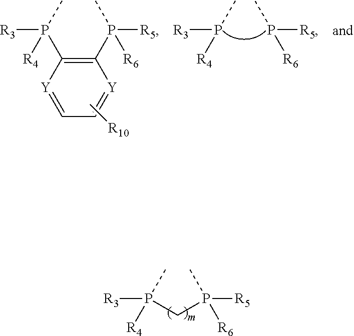
in which m is 1, 2, or 3;
R₉ is H, alkyl, or aryl;
R₁₀ is H, alkyl, aryl, or taken together with two of the atoms of the ring to which it is bound, forms a 6-membered ring;
Y is C or N;
L is a linker group; and
W is a water soluble group; or a pharmaceutically acceptable salt thereof.

2. The compound of claim 1, wherein the polycyclic moiety formed by R₁ and R₂, taken together with the gold (Au) to which they are bound, is selected from the group consisting of:

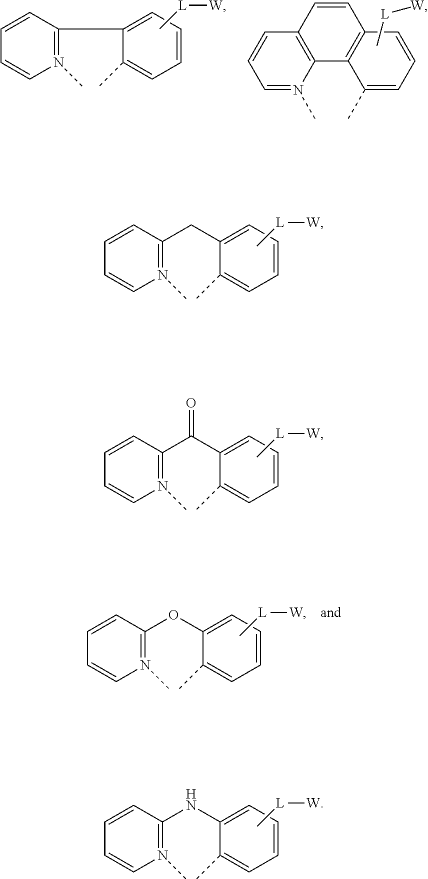

3. The compound of claim 1, having a structure selected from the group consisting of: 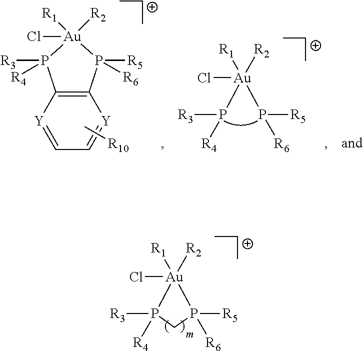
in which m is 1, 2, or 3;
wherein R₁ and R₂, taken together with the gold (Au) to which they are bound, form a polycyclic moiety selected from the group consisting of: 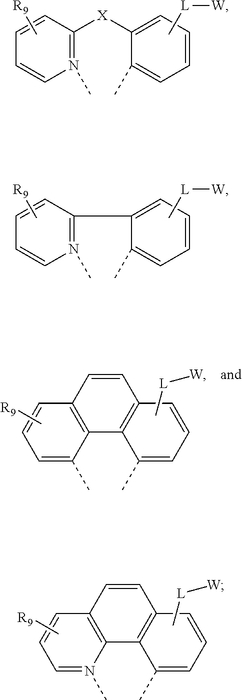
X is selected from the group consisting of CH₂, O, NH, C═O, C═NO-alkynyl, S, or aryl;
R₃, R₄, R₅, R₆ are each independently selected from the group consisting of H, alkyl, and aryl, or R₃ and R₄ and/or R₅ and R₆, taken together with the phosphorus (P) to which they are bound, form a cyclic moiety;
R₉ is H, alkyl, or aryl;
R₁₀ is H, alkyl, aryl, or taken together with two of the atoms of the ring to which it is bound, forms a 6-membered ring;
Y is C or N;
L is a linker group; and
W is a water soluble group; or a pharmaceutically acceptable salt thereof.

4. The compound of claim 1, having the structure: 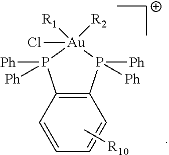
wherein R₁ and R₂, taken together with the gold (Au) to which they are bound, form a polycyclic moiety selected from the group consisting of: 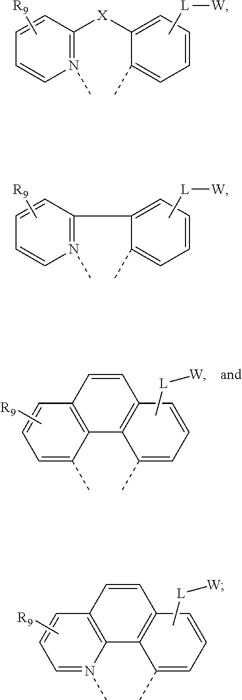
X is selected from the group consisting of CH₂, O, NH, C═O, C═NO-alkynyl, S, or aryl;
R₉ is H, alkyl, or aryl;
R₁₀ is H, alkyl, aryl, or taken together with two of the atoms of the ring to which it is bound, forms a 6-membered ring;
L is a linker group; and
W is a water soluble group; or a pharmaceutically acceptable salt thereof.

5. The compound of claim 1, having a structure selected from the group consisting of: 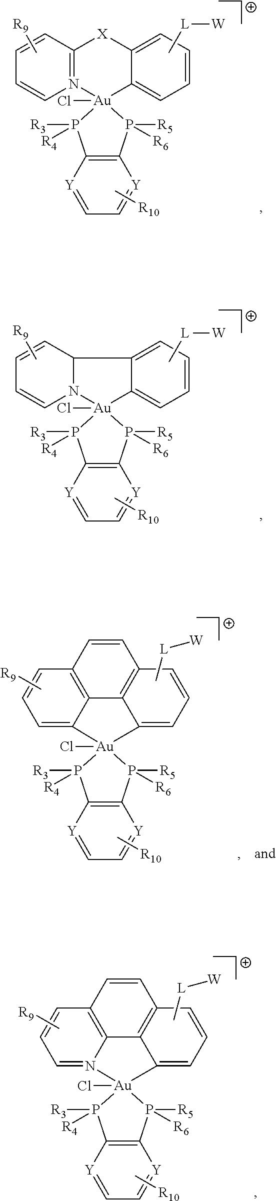
wherein
X is selected from the group consisting of CH₂, O, NH, C═O, C═NO-alkynyl, S, or aryl;
R₃, R₄, R₅, R₆ are each independently selected from the group consisting of H, alkyl, and aryl, or R₃ and R₄ and/or R₅ and R₆, taken together with the phosphorus (P) to which they are bound, form a cyclic moiety;
R₉ is H, alkyl, or aryl;
R₁₀ is H, alkyl, aryl, or taken together with two of the atoms of the ring to which it is bound, forms a 6-membered ring;
Y is C or N;
L is a linker group; and
W is a water soluble group; or a pharmaceutically acceptable salt thereof.

6. The compound of claim 1, having a structure: 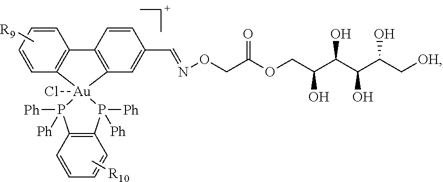
wherein R₉ is H, alkyl, or aryl; and R₁₀ is H, alkyl, aryl, or taken together with two of the atoms of the ring to which it is bound, forms a 6-membered ring.

7. The compound of claim 1, having a structure: 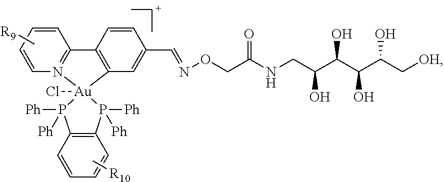
wherein R₉ is H, alkyl, or aryl; and R₁₀ is H, alkyl, aryl, or taken together with two of the atoms of the ring to which it is bound, forms a 6-membered ring.

8. The compound of claim 1, having a structure: 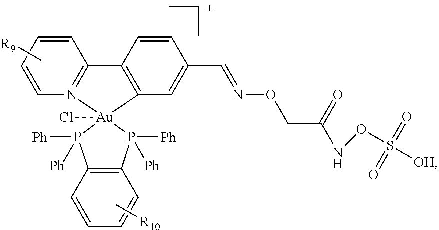
wherein R₉ is H, alkyl, or aryl; and R₁₀ is H, alkyl, aryl, or taken together with two of the atoms of the ring to which it is bound, forms a 6-membered ring.

9. The compound of claim 1, wherein W is selected from the group consisting of: 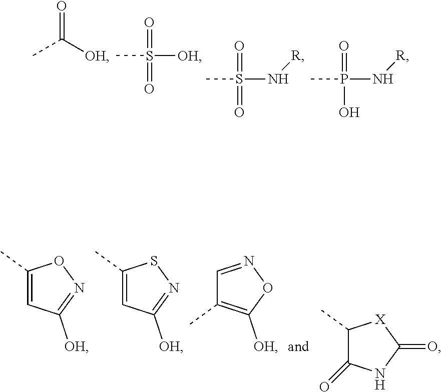
wherein R is selected from H, alkyl, and aryl.

10. The compound of claim 1, wherein W is selected from the group consisting of: 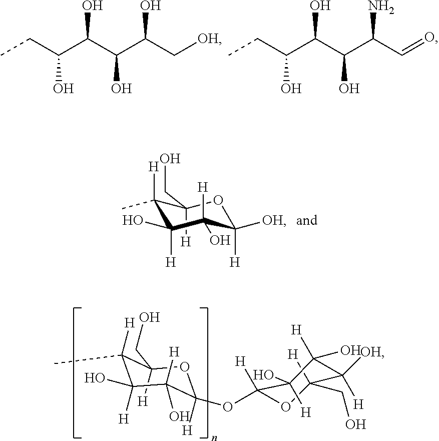
wherein n is 1-10.

11. The compound of claim 1, wherein W is selected from the group consisting of: 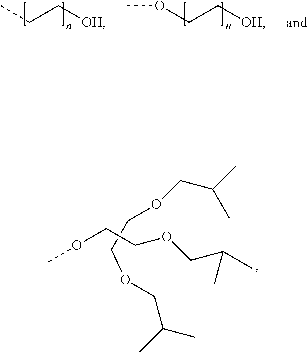
wherein n is 1-10.

12. The compound of claim 1, wherein L is selected from the group consisting of

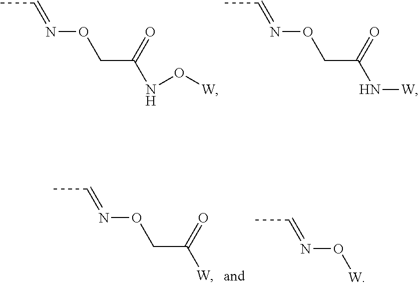

13. A pharmaceutical composition comprising the compound of claim 1 and a pharmaceutically-acceptable carrier.

14. A compound of the formula: 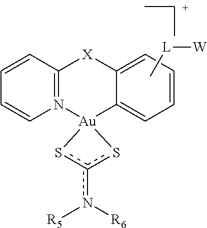
wherein
X is selected from the group consisting of CH₂, O, NH, C═O, C═NO-alkynyl, S, or aryl;
R₁ and R₂ are independently selected the group consisting of alkyl and substituted alkyl, or, R₁ and R₂, taken together with the N to which they are bound, form a piperidine or piperazine that is optionally substituted with Z;
Z is selected from the group consisting of haloaryl and alkoxyaryl;
L is a linker group; and
W is a water soluble group.

15. The compound of claim 14, wherein W is selected from the group consisting of: 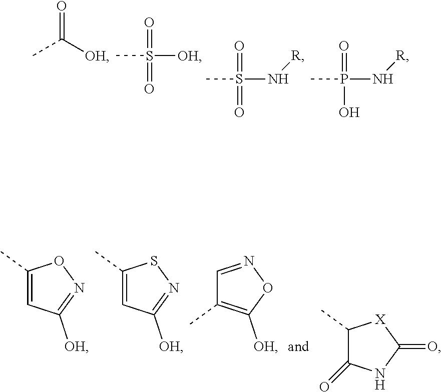
wherein R is selected from H, alkyl, and aryl; 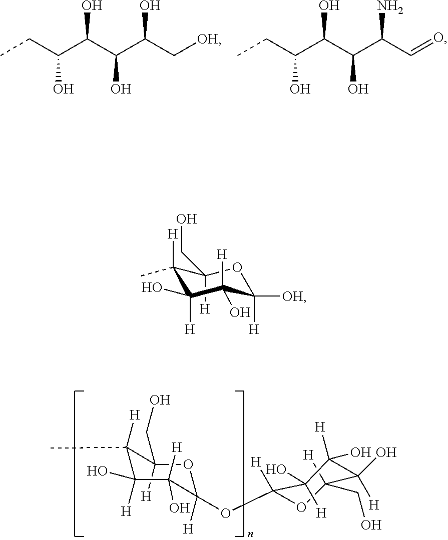
in which n is 1-10, 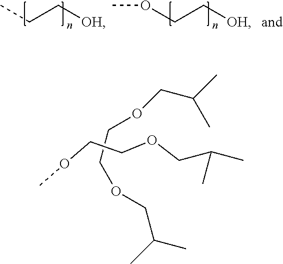
in which n is 1-10.

16. The compound of claim 1, wherein L is selected from the group consisting of

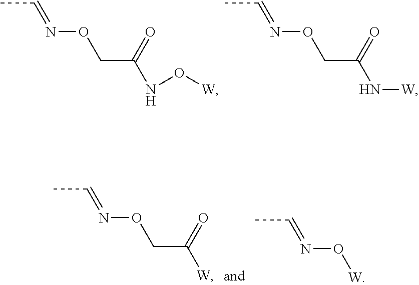

17. A pharmaceutical composition comprising the compound of claim 14 and a pharmaceutically-acceptable carrier.

18. A method of making the compound 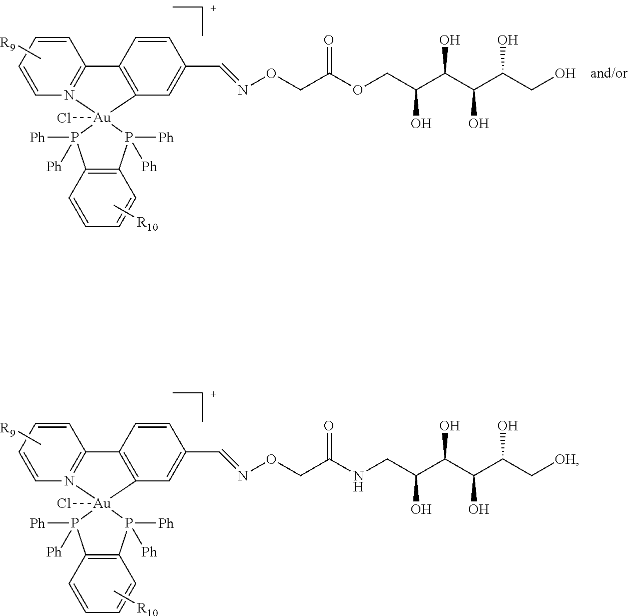
comprising reacting 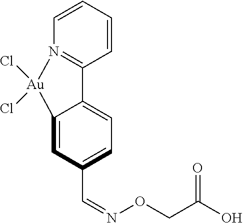
with sorbitol and/or glucamine in the presence of 1-Ethyl-3-(3-dimethylaminopropyl)carbodiimide hydrochloride (EDC), 1-hydroxybenzotriazole (HOBt), and anhydrous dimethylformamide (anh.DMF).

19. The method of claim 18, further comprising obtaining the compound 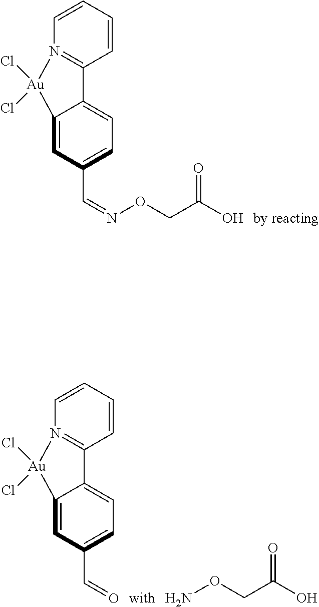
in the presence of HCl.

20. The method of claim 19, further comprising obtaining the compound 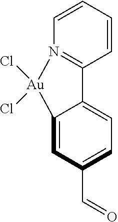
by reacting 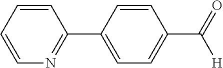 [SMILES: O=Cc1ccc(-c2ccccn2)cc1]
with HAuCl₄ in the presence of water.
